(19) United States
(12) Patent Application Publication
(10) Pub. No.: US 2010/0268074 A1
(43) Pub. Date: Oct. 21, 2010

(54) STRONTIUM HALIDE SCINTILLATORS, DEVICES AND METHODS

(75) Inventors: Edgar V. Van Loef, Allston, MA (US); Kanai S. Shah, Newton, MA (US); Jarek Glodo, Allson, MA (US); Cody M. Wilson, Winchester, MA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Radiation Monitoring Devices, Inc., Watertown, MA (US)

(21) Appl. No.: 12/497,436

(22) Filed: Jul. 2, 2009

Related U.S. Application Data
(60) Provisional application No. 61/077,826, filed on Jul. 2, 2008.
(60) Provisional application No. 61/094,796, filed on Sep. 5, 2008.

(51) Int. Cl. A61B 6/03; G01T 1/166; G01T 1/208; G01T 1/20; G01D 18/00; G21H 5/02; G01V 5/08; C09K 11/77; C09K 11/55; A61B 6/00

(52) U.S. Cl. 600/431; 250/363.04; 250/369; 250/362; 250/252.1; 250/302; 250/269.1; 378/19; 252/301.4 R

(57) ABSTRACT

The present invention provides strontium halide scintillators as well as related radiation detection devices, imaging systems, and methods.

### CROSS REFERENCES TO RELATED APPLICATIONS

The present application claims the benefit of priority under 35 U.S.C. §119(e) of U.S. Application No. 61/077,826, filed Jul. 2, 2008 (Attorney Docket No. 022071-003600US) and U.S. Application No. 61/094,796, filed Sep. 5, 2008 (Attorney Docket No. 022071-003610US), the entire contents of which are incorporated herein by reference.

### BACKGROUND OF THE INVENTION

The present invention relates to scintillator compositions and related devices and methods. More specifically, the present invention relates to scintillator compositions including a strontium halide composition and a dopant for use, for example, in radiation detection, including gamma-ray spectroscopy, and X-ray and neutron detection.

Scintillation spectrometers are widely used in detection and spectroscopy of energetic photons (e.g., X-rays, gamma-rays, etc.). Such detectors are commonly used, for example, in nuclear and particle physics research, medical imaging, diffraction, non destructive testing, nuclear treaty verification and safeguards, nuclear non-proliferation monitoring, and geological exploration.

Single photon emission computed tomography (SPECT), for example, is a powerful, noninvasive medical imaging modality that mathematically reconstructs the three dimensional distribution of a radionuclide throughout the body of a human patient or a research animal. Typically, the collected data are displayed and evaluated as a set of two-dimensional images through the organ or diseased area under investigation. SPECT allows quantitative and functional study in an investigated subject/region and therefore is an extremely useful tool for examination and/or study of dynamic physiological systems, such as organ and tissue physiology including that in the heart, lung, kidney, liver, brain, and skeletal system. SPECT agents now are becoming available for prostate and other forms of cancer as well. SPECT is very commonly used in identifying and localizing coronary artery disease and as many as 90% of all myocardial perfusion studies are now performed using SPECT.

The performance of SPECT systems often is limited by the detectors used in these systems. Modern SPECT systems include scintillation crystals coupled to photomultiplier tubes as detectors. Important requirements for scintillators used in SPECT applications include high light output and high energy resolution, reasonably fast response and high gamma ray stopping efficiency. Ideally, the scintillator should also be inexpensive, rugged and easy to manufacture. Currently, NaI(Tl) is the detector of choice in SPECT systems and it is relatively inexpensive and its light output is fairly large. However, the poor energy resolution of NaI(Tl) often limits SPECT performance. The energy resolution of NaI:Tl is limited by its relatively poor proportionality. If scintillators with higher energy resolution at typical SPECT energies (˜440 keV) were available, the essential process of scatter rejection would improve. Furthermore, dual-isotope imaging, which is a unique property of SPECT (compared to PET), would also become possible if scintillators with high energy resolution became available.

In the last five years, cerium doped lanthanum bromide (LaBr₃:Ce) has emerged as a promising scintillator for gamma-ray spectroscopy. LaBr₃:Ce and other related rare earth trihalides (such as CeBr₃) provide high light output (>60,000 photons/MeV) along with very fast response (≦20 ns). Correspondingly, the energy resolution of these materials is very high at 511 keV (˜3.5% FWHM), which is almost a factor of two higher than that from NaI:Tl. However, the energy resolution of LaBr₃:Ce (and related scintillators) at typical SPECT energy of 140 keV (^{99m}Tc) is more modest (˜7% FWHM using typical bialkali photocathode) and is only slightly better than that of NaI:Tl (˜9% FWHM at 140 keV). This is primarily because LaBr₃:Ce shows increased nonproportionality as the electron energy decreases, the effect of which is felt more acutely at lower γ-ray energies (where most SPECT isotopes emit). Thus, LaBr₃:Ce and related rare earth halides, which show very high timing resolution and high energy resolution at 511 keV, appear to be better suited for time-of-flight PET rather than for SPECT at present. The current cost of LaBr₃:Ce is also very high which is primarily due to difficulties (such as cracking and cleavage) associated with growth of large LaBr₃:Ce crystals which have anisotropic, hexagonal crystal structure. As a result, the search for improved scintillators for SPECT continues.

Important requirements for the scintillation crystals used in these applications, including SPECT, include high light output, transparency to the light it produces, high stopping efficiency, fast response, good proportionality and energy resolution, low cost, and availability in large volume. These requirements on the whole cannot be met by many of the commercially available scintillator compositions. While general classes of chemical compositions may be identified as potentially having some attractive scintillation characteristic(s), specific compositions/formulations having both scintillation characteristics and physical properties necessary for actual use in scintillation spectrometers and various practical applications have proven difficult to predict. Specific scintillation properties are not necessarily predictable from chemical composition alone, and preparing effective scintillator compositions from even candidate materials often proves difficult. For example, while the composition of sodium chloride had been known for many years, the invention by Hofstadter of a high light-yield and conversion efficiency scintillator from sodium iodide doped with thallium launched the era of modern radiation spectrometry. More than half a century later, thallium doped sodium iodide, in fact, still remains one of the most widely used scintillator materials. Since the invention of NaI(Tl) scintillators in the 1940's, for half a century radiation detection applications have depended to a significant extent on this material. The fields of nuclear medicine, radiation monitoring, and spectroscopy have grown up supported by NaI(Tl). Although far from ideal, NaI(Tl) was relatively easy to produce for a reasonable cost and in large volume. With the advent of X-ray CT in the 1970's, a major commercial field emerged as did a need for different scintillator compositions, as NaI(Tl) was not able to meet the requirements of CT imaging. Later, the commercialization of positron emission tomography (PET) imaging provided the impetus for the development of yet another class of detector materials with properties suitable for PET. As the methodology of scintillator development evolved, new materials have been added, and yet, specific applications are still hampered by the lack of scintillators suitable for particular applications.

As a result, there is continued interest in the search for new scintillator compositions and formulations with both the enhanced performance and the physical characteristics needed for use in various applications, one of them being single photon emission computed tomography (SPECT). Today, the development of new scintillator compositions continues to be as much an art as a science, since the composition of a given material does not necessarily determine its properties as a scintillator, which are strongly influenced by the history (e.g., fabrication process) of the material as it is formed. While it may be possible to reject a potential scintillator for a specific application based solely on composition, it is typically difficult to predict whether even a material with a promising composition can be used to produce a useful scintillator with the desired properties.

A need exists for improved scintillator compositions, as well as scintillator based devices and systems suitable for use in various radiation detection applications, including medical imaging applications.

### BRIEF SUMMARY OF THE INVENTION

The present invention provides strontium halide scintillators as well as related radiation detection devices and imaging systems.

In one aspect, the present invention provides a strontium halide scintillator composition. Scintillator compositions can further include a dopant, such as europium. In one embodiment, a scintillator composition includes a europium doped strontium iodide composition.

Scintillator compositions are suitable for use in various imaging and/or radiation detection devices and systems, as well as imaging methods. In one embodiment, an imaging system includes a subject area; a radiation detection assembly including a strontium halide (e.g., SrI₂:Eu) scintillator material and a photodetector assembly optically coupled to the scintillator material; and electronics coupled to the radiation detection assembly so as to output image data in response to radiation detected by the scintillator. Imaging systems and methods can include computed tomography, such as single photon emission computed tomography (SPECT).

For a fuller understanding of the nature and advantages of the present invention, reference should be made to the ensuing detailed description taken in conjunction with the accompanying drawings. The drawings represent embodiments of the present invention by way of illustration. The invention is capable of modification in various respects without departing from the invention. Accordingly, the drawings/figures and description of these embodiments are illustrative in nature, and not restrictive.

### BRIEF DESCRIPTION OF THE DRAWINGS

FIG. 1 illustrates a basic schematic diagram of a radiation detection assembly of the present invention.

FIG. 2 illustrates a basic diagram of a SPECT imaging system, according to an embodiment of the present invention.

FIG. 3 shows a schematic of a positron emission scanner system.

FIG. 4 shows a schematic of a detector arrangement for a positron emission scanner system.

FIG. 5 shows a schematic of an x-ray computed tomography scanner system, according to an embodiment of the present invention.

FIG. 6 shows an X-ray excited optical emission spectrum of SrI₂:Eu crystal.

FIG. 7 illustrates a decay time spectrum of a SrI₂:Eu scintillator material.

FIG. 8 illustrates a beta-excited radioluminescence spectrum acquired of a Eu-doped strontium iodide sample.

FIG. 9 illustrates time-resolved luminescence decay acquired in one example by excitation with 30 ns laser pulses at 266 nm.

FIG. 10 shows a pulse height spectrum acquired of one SrI₂(0.5% Eu) crystal sample yielding an energy resolution of 3.7% at 662 keV.

FIG. 11 shows pulse-height spectra acquired with Ba-133, Am-241, Co-57, Na-22, Co-60 and Cs-137 sources provide the energy resolution as a function of gamma ray energy. Energy resolution is comparable between LaBr₃(Ce) and SrI₂(Eu) for all energies.

FIG. 12 illustrates relative light yields as a function of electron energy acquired using the SLYNCI, which reveal that SrI₂(Eu) has fairly proportional light yield in the 4-440 keV range, in comparison to both LaBr₃(Ce) and NaI(Tl). This result suggests that the energy resolution for SrI₂(Eu) has potential for improvement over that measured so far, by improving crystal uniformity and optical quality, and with an optimized reflector assembly.

FIG. 13 illustrates radioluminescence spectra for strontium halide compositions, according to embodiments of the present invention.

FIG. 14 illustrates scintillation decay time spectra for strontium halide compositions, according to embodiments of the present invention.

FIG. 15 illustrates afterglow for strontium halide composition at longer time scales, according to an embodiment of the present invention.

FIG. 16 illustrates pulse height spectrum of ²⁴¹Am and that of the single photoelectron.

FIG. 17 illustrates light output for strontium halide compositions, according to embodiments of the present invention.

FIG. 18 illustrates energy spectra for strontium halide compositions, according to embodiments of the present invention.

FIG. 19 illustrates relative light yield as a function of gamma ray energy for strontium iodide doped with Eu, according to an embodiment of the present invention.

FIGS. 20A-20C provides histograms showing radioluminescence (FIG. 20A), proportionality (FIG. 20B), and decay time (FIG. 20C) for SrI₂ scintillator material doped with thallium (Tl).

FIGS. 21A-21C provide histograms showing decay time (FIG. 21A); radioluminescence (FIG. 21B); and energy spectrum showing energy resolution for SrI₂ scintillator material doped with thallium (Tl).

FIG. 22 illustrates light output of SrI2(Eu) as a function of temperature in the range of 25 degrees C. to 175 degrees C. As shown, light output increases with temperature in the examined range.

### DETAILED DESCRIPTION OF THE INVENTION

The present invention provides strontium halide scintillators as well as related radiation detection devices and imaging systems. A composition of strontium iodide doped with europium was initially discovered over 40 years ago as having scintillation properties. Europium activated strontium iodide compositions are described, for example, in U.S. Pat. No. 3,373,279. Unfortunately, while properties such as light output were studied, investigation and further development of such materials has been limited and the material received little attention following its discovery, with little practical use previously being recognized for this composition.

This invention will be better understood with resort to the following definitions:

Rise time, in reference to a scintillation crystal material, shall mean the speed with which its light output grows once a gamma-ray has been stopped in the crystal. The contribution of this characteristic of a scintillator combined with the decay time contribute to a timing resolution.

A Fast timing scintillator (or fast scintillator) typically includes a timing resolution of about 500 ps or less. For certain PET applications (e.g., time-of-flight (TOF)), the fast scintillator should be capable of localizing an annihilation event as originating from within about a 30 cm distance, i.e., from within a human being scanned. Thus, TOF PET imaging applications typically require a fast timing scintillator having a timing resolution of about 500 ps or less.

Timing accuracy or resolution, usually defined by the full width half maximum (FWHM) of the time of arrival differences from a point source of annihilation gamma-rays. Because of a number of factors, there is a spread of measured values of times of arrival, even when they are all equal. Usually they distribute along a bell-shaped or Gaussian curve. The FWHM is the width of the curve at a height that is half of the value of the curve at its peak.

Light Output shall mean the number of light photons produced per unit energy deposited by the detected gamma-ray, typically the number of light photons/MeV.

Stopping power or attenuation shall mean the range of the incoming X-ray or gamma-ray in the scintillation crystal material. The attenuation length, in this case, is the length of crystal material needed to reduce the incoming beam flux to 1/e⁻.

Proportionality of response (or linearity). For some applications (such as CT scanning) it is desirable that the light output be substantially proportional to the deposited energy. For applications such as spectroscopy, non-proportionality of response is an important parameter. In a typical scintillator, the number of light photons produced per MeV of incoming gamma-ray energy is not constant. Rather, it varies with the energy of the stopped gamma-ray. This has two deleterious effects. The first is that the energy scale is not linear, but it is possible to calibrate for the effect. The second is that it degrades energy resolution. To see how this occurs, consider a scintillator that produces 300 photons at 150 keV, 160 photons at 100 keV and 60 photons at 50 keV. From statistics alone, the energy resolution at 150 keV should be the variability in 300 photons, which is 5.8%, or 8.7 keV. If every detected event deposited 150 keV in one step this would be the case. On the other hand, if, as it occurs, an event deposited 100 keV in a first interaction and then another 50 keV in a second interaction, the number of photons produced would not be 300 on the average, but 160+60=220 photons, for a difference of 80 photons or 27%. In multiple detections, the peak would broaden well beyond the theoretical 8.7 keV. The smaller the non-proportionality the smaller this broadening and the closer the actual energy resolution approaches the theoretical limit.

As described herein, the present invention is based at least partially on the discovery of strontium halide materials, such as strontium iodide doped with europium, surprisingly having certain previously unidentified scintillation characteristics including, for example, a very high linearity of response as well as a very high light output, which together lead to very high energy resolution. These previously undiscovered strontium halide compositions with such properties make the described compositions suitable for various uses previously unrecognized, such as certain imaging applications (e.g., computed tomography imaging, SPECT, PET). Furthermore, such energy resolution in a material that is relatively easy to grow in crystal form, permits its incorporation in detection devices that realize not just quantitative improvements in performance, but qualitative gains as well. One exemplary application includes dual energy imaging with single photon emission computed tomography (SPECT) scintillation cameras. The strontium halide compositions of the invention further demonstrated surprisingly robust/fast decay time properties that were previously uncharacterized, making the compositions suitable for previously unrecognized uses including imaging techniques such as computed tomography imaging (e.g. X-ray CT imaging, SPECT, PET).

New scintillator materials with high light output, excellent proportionality, very high energy resolution and fast response would offer unique advantages over many of the existing scintillators used in γ-ray studies. One application addressed herein is SPECT, where the proposed scintillators would offer better scatter rejection and possibility of dual isotope imaging. Furthermore, scintillators with high light output provide other practical benefits: larger PMTs can be used without any degradation in spatial resolution, which can significantly reduce the SPECT system cost. In addition to clinical SPECT systems and gamma-cameras, surgical probes, small animal imaging systems, and dedicated organ imaging systems would all benefit from the proposed innovation. Due to their high light output, SrI₂:Eu scintillators can be used with solid-state photodetectors such as Si p-i-n photodiodes and avalanche photodiodes in place of PMTs. These photodiodes generally have higher noise than PMTs, so high light output of the new scintillators is essential in ensuring that the overall system performance is not degraded by the photodiode noise. These silicon photodiodes provide some important benefits such as compactness, ruggedness and higher quantum efficiency. This would allow development of compact, portable as well as flexible instrumentation without compromise in performance. Furthermore, insensitivity of silicon photodiodes to magnetic fields can be exploited to develop compact SPECT-MR systems based on SrI₂:Eu scintillators with photodiode readout.

These SrI₂:Eu scintillators also have critical applications in other areas. The increased interest and commitment to quality control has motivated many industrial groups to develop γ-ray based nondestructive testing equipment. High energy resolution, wide dynamic range, high sensitivity, and low noise performance are important in these applications. This is an area in which the compactness, and flexibility of a high performance detector is have a major impact. Other applications include homeland security studies, nuclear physics research, nuclear treaty verification, environmental monitoring, nuclear waste clean-up, astronomy and well-logging. In homeland security monitoring, it is important to have scintillators that do not have any self-activation. This is particularly important when the detector volume is large and the expected extrinsic activity is very small. In these situations, self activity of LaBr₃:Ce can be problematic. In LaBr₃, self-activity is primarily due to ¹³⁸La that emits conversion electrons and β-particles with energy of up to 1.7 MeV. The self-activity due to ¹³⁸La in LaBr₃ has an intrinsic count-rate of ˜1.5 events/(cm³·sec). SrI₂:Eu has virtually no self-activity, which makes it more attractive in homeland security monitoring.

In another aspect, strontium halide scintillators of the present invention can be used for radiation detection at elevated or high temperatures. Strontium halide scintillators, such as strontium iodide containing compositions, demonstrate surprisingly high light output at high temperatures. Thus, the unexpected characteristic of strontium halide scintillators (e.g., SrI2) having excellent light output at high temperature, makes the scintillator compositions of the present invention suitable for high temperature radiation detection applications, such as well logging.

Scintillator compositions of the present invention include strontium halide compounds, typically doped with one or more dopants. Strontium halide compositions can include a single halide (e.g., SrI) or strontium and a mixture of two or more halides. Compositions can include a dopant, which can include a single dopant or mixture of dopants. Different compounds within the scope of the invention compositions may have different scintillation characteristics, and a particular composition ratio or formulation selected may be at least partially based on intended use of the scintillator composition and/or desired properties or scintillation/performance characteristics.

As described above, scintillator composition of the present invention can optionally include a “dopant”. Dopants can affect certain properties, such as physical properties (e.g., brittleness, etc.) as well as scintillation properties (e.g., luminescence, etc.) of the scintillator composition. Exemplary dopants include, for example, cerium (Ce), europium (Eu), thallium (Tl), Sodium (Na), and the like, as well as mixtures of two or more dopants. The amount of dopant present will depend on various factors, such as the application for which the scintillator composition is being used; the desired scintillation properties (e.g., emission properties, timing resolution, etc.); and the type of detection device into which the scintillator is being incorporated. For example, the dopant is typically employed at a level in the range of about 0.1% to about 20%, by molar weight. In certain embodiments, the amount of dopant is in the range of about 0.1% to less than about 100% (including any value therebetween), or about 0.1% to about 5.0%, or about 5.0% to about 20%, by molar weight.

The scintillator compositions of the invention may be prepared in several different forms. In some embodiments, the composition is in a crystalline form (e.g., monocrystalline). Scintillation crystals, such as monocrystalline scintillators, have a greater tendency for transparency than other forms. Scintillators in crystalline form (e.g., scintillation crystals) are often useful for high-energy radiation detectors, e.g., those used for gamma-ray or X-ray detection. However, the composition can include other forms as well, and the selected form may depend, in part, on the intended end use of the scintillator. For example, a scintillator can be in a powder form. It can also be prepared in the form of a ceramic or polycrystalline ceramic. Other forms of scintillation compositions will be recognized and can include, for example, glasses, deposits, vapor deposited films, microcolumnar, or other forms suitable for radiation detection as described herein. It should also be understood that a scintillator composition might contain small amounts of impurities. Also, minor amounts of other materials may be purposefully included in the scintillator compositions to affect the properties of the scintillator compositions.

Methods for making crystal materials can include those methods described herein and may further include other techniques. Typically, the appropriate reactants are melted at a temperature sufficient to form a congruent, molten composition. The melting temperature will depend on the identity of the reactants themselves (see, e.g., melting points of reactants), but is usually in the range of about 520-560° C. Non-limiting examples of the crystal-growing methods can include certain techniques of the Bridgman-Stockbarger methods; the Czochralski methods, the zone-melting methods (or “floating zone” method), the vertical gradient freeze (VGF) methods, and the temperature gradient methods. See, e.g., Example 1 infra. (see also, e.g., “Luminescent Materials”, by G. Blasse et al, Springer-Verlag (1994) and “Crystal Growth Processes”, by J. C. Brice, Blackie & Son Ltd (1986)).

In the practice of the present invention, attention is paid to performance/scintillation characteristics as well as the physical properties of the scintillator material. In particular applications, properties such as hygroscopy (tendency to absorb water), brittleness (tendency to crack), and crumbliness should be minimized. Certain scintillation characteristics measured in exemplary embodiments are set forth below in Table 1. Listed characteristics are provided by way of example, not limitation, as further improvement may be expected.

TABLE 1
Scintillator Properties

| | | Light Output | Energy | Emission | Rise- | | |
|---|---|---|---|---|---|---|---|
| Scintillator | | (Photons/ | Resolution | Range | time | Decay Time | Non- |
| Composition | Z_{eff} | MeV) | (662 keV) | (nm) | (ns) | (ns) | Proportionality |
| LaBr₃:Ce | 45.7 | 63,000 | 2.8% | ~325-425 | | 15 (97%), | 4% (60-1274 keV) |
| | | | | | | 66 (3%) | |
| SrI₂:0.5% Eu | 50 | 68,000 | 5.3% | ~400-460 | <2 | ~620 | 4.8% |
| SrI₂:2% Eu | ″ | 84,000 | 3.9% | ″ | ″ | ~900 | 6.2% |
| SrI₂:5% Eu | ″ | 120,000 | 2.8% | ″ | ″ | ~1,100 | 2.0% |
| SrI₂:8% Eu | ″ | 80,000 | 4.9% | ″ | ″ | | 5.1% |
| SrI₂:10% Eu | ″ | | | ″ | ″ | ~1650 | |
| SrI₂:0.5% Ce/Na | ″ | 16,000 | 6.4% | ~350-475 | 2.5 | ~270; | 8% (60-1274 keV) |
| | | | | | | 25 (47%), | |
| | | | | | | 159 (53%) | |
| SrI₂:2% Ce/Na | ″ | 11,000 | 12.3% | ” | | 32 (46%), | 6% (60-1274 keV) |
| | | | | | | 450 (53%) | |

Table 1 provides a listing of certain measured properties of a number of exemplary strontium halide compositions of the present invention (see also, e.g., Examples below). Previously known LaBr₃:Ce is included for comparison (light output and energy resolution is as quoted by Saint Gobain). Non-proportionality is measured over the range of 14 keV to 1274 keV unless otherwise noted.

Characteristics of the scintillator compositions of the present invention include robust light output, good proportionality and energy resolution, and/or fast response. In one embodiment, scintillation properties of properties of strontium halide compositions included a peak emission wavelength that is well matched to PMTs as well as silicon diodes used in many detection and imaging systems. Scintillator compositions of the present invention include scintillators with rapid rise time and relatively fast decay-time constants. Rise time of the scintillator compositions will typically be less than about 5 ns, and more typically less than about 3 ns (e.g., about 1 ns to about 3 ns), and even less than 2 ns. Decay time constant will typically be in a range of about 1-2000 ns, including less than about 50, 100, 300, 500, 1000, or 2000 ns. Scintillators will typically include a light output greater than about 10,000 photons/Mev, 30,000, 50,000, 80,000 photons/MeV, and more typically greater than about 100,000 photons/MeV. Energy resolution will typically be in a range of about 3-15% at 662 keV, and more typically between about 3-10%, including better than or less than about 3%, 5%, 8%, or 10% at 662 keV.

As set forth above, scintillator compositions of the present invention may find use in a wide variety of applications. In one embodiment, for example, the invention is directed to a method for detecting energy radiation (e.g., gamma-rays, X-rays, neutron emissions, and the like) with a scintillation detector including the scintillation composition of the invention.

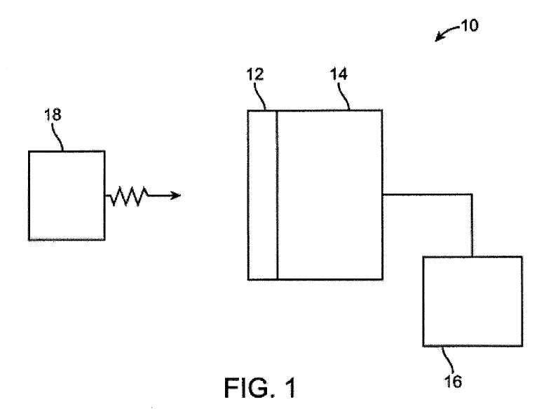

FIG. 1 is a schematic diagram of a detector assembly of the present invention. The detector 10 includes a scintillator 12 optically coupled to a light photodetector 14 or imaging device. The detector assembly 10 can include a data analysis, or computer control system 16 to process information from the scintillator 12 and light photodetector 14. In use, the detector 10 detects energetic radiation emitted form a source 18. The detector assembly can be included, in whole or in part, in detector and imaging systems, e.g., as described further below.

A data analysis and/or computer system thereof can include, for example, a module or system to process information (e.g., radiation detection information) from the detector/photodetectors can also be included in an invention assembly and can include, for example, a wide variety of proprietary or commercially available computers, electronics, or systems having one or more processing structures, a personal computer, mainframe, or the like, with such systems often comprising data processing hardware and/or software configured to implement any one (or combination of) the method steps described herein. Any software will typically comprise machine readable code of programming instructions embodied in a tangible media such as a memory, a digital or optical recording media, optical, electrical, or wireless telemetry signals, or the like, and one or more of these structures may also be used to transmit data and information between components of the system in any of a wide variety of distributed or centralized signal processing architectures.

The detector assembly typically includes material formed from the scintillator composition described herein (e.g., one or more scintillator crystals). The detector further can include, for example, a light detection assembly including one or more photodetectors. Non-limiting examples of photodetectors include photomultiplier tubes (PMT), photodiodes, CCD sensors, image intensifiers, and the like. Choice of a particular photodetector will depend in part on the type of radiation detector being fabricated and on intended use of the device. In certain embodiments, the photodetector may be position-sensitive.

The detector assemblies themselves, which can include the scintillator and the photodetector assembly, can be connected to a variety of tools and devices, as mentioned previously. Non-limiting examples include nuclear weapons monitoring and detection devices, well-logging tools, and imaging devices, such as nuclear medicine devices (e.g., SPECT, PET, x-ray CT). Various technologies for operably coupling or integrating a radiation detector assembly containing a scintillator to a detection device can be utilized in the present invention, including various known techniques. The detectors may also be connected to a visualization interface, imaging equipment, or digital imaging equipment (e.g., pixilated flat panel devices).

Imaging devices, including medical imaging equipment, such as the PET and SPECT devices, and the like (e.g., discussed further below), represent an important application for invention scintillator compositions and radiation detectors. Furthermore, geological exploration devices, such as well-logging devices, were mentioned previously and represent an important application for these radiation detectors. The assembly containing the scintillator usually includes, for example, an optical window at one end of the enclosure-casing. The window permits radiation-induced scintillation light to pass out of the scintillator assembly for measurement by the photon detection assembly or light-sensing device (e.g., photomultiplier tube, etc.), which is coupled to the scintillator assembly. The light-sensing device converts the light photons emitted from the scintillator into electrical pulses that may be shaped and digitized, for example, by the associated electronics. By this general process, gamma-rays can be detected, which in turn provides an analysis of geological formations, such as rock strata surrounding the drilling bore holes.

In many of the applications of a scintillator composition as set forth above (e.g., nuclear weapons monitoring and detection, imaging, and well-logging technologies), certain characteristics of the scintillator are desirable, including high light output, fast rise time and short decay time, good timing resolution, and suitable physical properties. The present invention is expected to provide scintillator materials that can provide the desired high light output and initial photon intensity characteristics for demanding applications of the technologies. Moreover, the invention scintillator compositions are also expected to simultaneously exhibit the other important properties noted above, e.g., short decay time and good energy resolution. Furthermore, the scintillator materials are also expected to be produced efficiently and economically, and also expected to be employed in a variety of other devices which require radiation/signal detection (e.g., gamma-ray, X-ray, neutron emissions, and the like).

### Imaging Systems and Applications

Scintillator composition as described herein are well suited for various imaging applications, including SPECT imaging. Strontium halide compositions, such as SrI₂, belong to the alkaline earth halide family and has orthorhombic structure. Crystals of doped strontium halide (e.g., SrI₂) compositions were grown, e.g., by Bridgman method, and their scintillation properties were measured. The optical emission from various SrI₂ compositions was measured and peak emission ranges were detected that are well suited and matched with many existing photodetection components Compositions further included suitable time profiles, including fast rise and decay times. The light output of the grown SrI₂ crystal was measured and remarkably high for various compositions. In some embodiments, light output was more than two times higher than that for NaI:Tl, the traditional scintillator used in SPECT and at least 30% higher than the LaBr₃:Ce crystal used in comparison studies. The energy resolution of SrI₂:Eu crystal was also excellent, e.g., ˜3.7% (FWHM) at 662 keV in one example and about two times better than NaI:Tl and approaching that for LaBr₃:Ce.

The proportionality of SrI₂ was measured to be excellent under gamma-ray and electron exposures. Particularly encouraging was the higher proportionality of SrI₂ at low electron energies (compared to even LaBr₃:Ce). This result indicates that once high optical quality, uniform SrI₂ crystals become available, their energy resolution for typical radioisotopes (^{99m}Tc, ²⁰¹Tl, ¹²³I, ¹¹¹In etc.) used in SPECT (gamma-emissions in 80-250 keV range) should be excellent. Already, the energy resolution of SrI₂:Eu, e.g., has been measured to be better than that for LaBr₃:Ce at 122 keV (⁵⁷Co source). Since the proportionality of SrI₂ crystals is excellent, their energy resolution ultimately should be dictated by photoelectron statistics. High quality strontium halide crystals should yield energy resolution of <5% (FWHM) at 140 keV for standard PMT read-out, approximately 2-fold better than NaI:Tl under similar conditions. This would allow strontium halide compositions to provide excellent scatter rejection capabilities and also enable dual-isotope SPECT studies.

Also, SrI₂ (with orthorhombic symmetry) does not have a layered crystal structure, which is the case for some compositions with hexagonal-type crystal structures (such as CaI₂, PbI₂, LuI₃ etc.). Hence, SrI₂ would be expected to be much less prone to basal cleavage. Alkaline earth halides such as SrI₂ are also less susceptible to oxyhalide formation, a common problem for rare earth halides such as LaBr₃. These considerations in addition to congruent melting of SrI₂ at low temperature (about 540° C.), and preliminary testing (see, e.g., as described herein) indicates grow large crystals of this exciting scintillator in a cost-effective manner from the melt using the Bridgman and Czochralski methods is possible. Based at least partially on these considerations, SrI₂ scintillator compositions as described is very attractive for SPECT.

Detector for Single Photon Imaging

The general detector requirements for SPECT are as generally recognized and can include considerations of patient safety and image quality. Patient safety requires that the detectors be sufficiently sensitive to optimize the use of the emitted radiation in order to minimize the patient dose while offering image quality sufficient for the diagnostic task. The specifications are derived from the characteristic of the isotopes used. Since SPECT is commonly performed (˜70% of the time) using ^{99m}Tc (140 keV), good detection efficiency and high energy resolution at 140 keV are needed, although radioisotopes with lower as well as higher energy emissions are also available. The detection efficiency typically must exceed 80% for the gamma ray energy of interest. Regarding energy resolution, the detector should be able to distinguish photoelectric events from Compton events. Typically, ˜9-10% (FWHM) energy resolution is obtained with NaI(Tl) crystals coupled to photomultipliers at 140 keV; however, better energy resolution would offer superior scatter rejection. Recent studies have shown that energy resolution ˜5% (FWHM) or better would provide adequate scatter rejection for imaging of dynamically moving target components, such as in myocardial perfusion studies. Estimates for the strontium halide compositions based on measured properties indicate that this scintillator should be able to achieve the target of ˜5% (FWHM) or better energy resolution at 140 keV. Count-rate requirements for SPECT are moderate and should be achievable with strontium halide compositions. Thus, strontium halide scintillator compositions can be utilized in methods and systems for dynamic imaging, such as in myocardial perfusion analysis or study.

Considerations for Dual Isotope Imaging

In yet another embodiment, strontium halide scintillator compositions can be utilized in methods and systems for dual isotope imaging, such as dual isotope SPECT imaging. The strontium halide compositions offer significantly increased brightness and highly proportional response in comparison to other materials, which are fundamental characteristics that improve signal-to-noise and the energy resolution performance of the detector. This directly improves scatter rejection and contrast resolution, which have their greatest significance for planar scintigraphy and SPECT for the detection of small or subtle changes in radionuclide uptake, and in improving the accuracy of radionuclide quantification. This can improve hot spot detection, for example in cancer imaging, cold spot discrimination needed for myocardial perfusion imaging, and for all applications of single-photon radionuclide imaging.

The high energy resolution expected for the strontium halide compositions also has important implications for dual-isotope imaging including dynamic imaging applications, such as ²⁰¹Tl/^{99m}Tc-sestamibi imaging of myocardial perfusion. Such dual isotope studies are performed sequentially by acquiring a ²⁰¹Tl myocardial perfusion scan at rest, followed by a myocardial perfusion scan acquired under stress with ^{99m}Tc-sestamibi. This provides an alternative to traditional myocardial imaging in which ²⁰¹Tl is injected to acquire a stress image, followed by a 2-3 hr redistribution period after which a ²⁰¹Tl rest image is acquired. Although the ²⁰¹Tl rest/stress study can be acquired with a single injection, the sequential ²⁰¹Tl/^{99m}Tc study shortens the procedure time and takes advantage of the improved photon statistics and photon energy characteristics from ^{99m}Tc-sestamibi for the stress scan.

Several previous efforts have investigated the feasibility of a clinical protocol in which ²⁰¹Tl and ^{99m}Tc-sestamibi rest/stress studies could be acquired simultaneously in a single imaging procedure. A simultaneous dual isotope stress/rest study could reduce camera time by half, thereby enhancing patient comfort, reducing patient motion artifacts, and improving throughput in comparison to the time needed to acquire two sequential studies. In addition, the dual isotope study would improve the geometrical alignment of the rest and stress images which are compared to differentiate viable ischemic tissue from infarct. The potential advantages of dual-isotope imaging have historically been offset by some important limitations. Specifically, in simultaneous dual-isotope imaging, the ²⁰¹Tl image is contaminated when γ-rays from ^{99m}Tc-sestamibi (140 keV) interact in the patient and are down-scattered into the ²⁰¹Tl energy window (70-80 keV), and when primary or scattered ₇-rays from the patient interact in the collimator and produce lead fluorescence x-rays. Previously reported Monte Carlo studies have shown that both ^{99m}Tc down-scatter and lead fluorescence x-rays overlap with the primary photons from ²⁰¹Tl. This represents a significant source of error in simultaneous dual-isotope ²⁰¹Tl and ^{99m}Tc-sestamibi imaging, with the lead x-rays making a 25% contribution to the contamination in the ²⁰¹Tl window and with the ^{99m}Tc cross-talk contamination representing 27% of total events in the ²⁰¹Tl window. Contamination of the ²⁰¹Tl image data can degrade image contrast, reduce geometric sharpness, and can frustrate radionuclide quantification. Several software techniques have been developed to compensate for the effects of cross-talk in simultaneous ²⁰¹Tl/^{99m}Tc imaging, including those implemented in iterative reconstruction techniques. In previously reported phantom experiments, software correction of simultaneously acquired dual-isotope rest ²⁰¹Tl and stress ^{99m}Tc SPECT images have shown similar myocardial-to-defect count ratios, defect sizes, and visual appearance in comparison to single isotope (²⁰¹Tl and ^{99m}Tc) SPECT images. Simultaneous ²⁰¹Tl/^{99m}Tc imaging also has been previously tested experimentally in a canine model of myocardial perfusion, and has been evaluated in a clinical setting. However, simultaneous ²⁰¹Tl/^{99m}Tc myocardial perfusion imaging still is not performed routinely in a clinical setting and improved methods of compensating cross-talk errors in combined dual-isotope techniques are desired. The improved energy resolution expected from the present strontium halide compositions has the potential of reducing errors due to contamination of the ²⁰¹Tl data from Pb x-ray and Compton scatter in dual-isotope ²⁰¹Tl/^{99m}Tc imaging. Thus, in one aspect, the present invention can include dual isotope imaging methods and systems including use of strontium halide scintillator compositions as described herein.

Other important examples of dual isotope imaging include the potential to assess multiple functions within the myocardium. Beyond the previous rest/stress perfusion studies described above, other functional studies are possible using dual-isotope studies with ^{99m}Tc (140 keV) and ¹²³I (159 keV). Some exemplary current myocardial perfusion agents labeled with ^{99m}Tc include sestamibi, teboroxime, and tetrafosmin, and can be imaged simultaneously with agents labeled with ¹²³I for fatty-acid metabolism or myocardial innervation (¹²³I-metaiodobenzylguanidine), or perfusion (¹²³I-iodorotenone). In addition, investigators at UCSF are developing an ¹²³I-labeled myocardial perfusion agent that exhibits uptake more linear with myocardial flow, higher myocardial extraction, and lower hepatic accumulation than other single-photon myocardial perfusion agents. In addition, the shorter half-life of ¹²³I allows higher levels of radioactivity to be injected to produce images with lower noise than ²⁰¹Tl. However, ¹²³I emits a high-energy photon from contaminants that similarly can scatter within the body or the detector to form a broad energy spectrum within the photopeak. By making use of the improved energy resolution that is expected from the proposed scintillators, the iodine-123 photo peak data can be acquired with narrower energy windows to improve contrast and quantification accuracy when these radionuclides are acquired either as single isotopes or in dual isotope imaging studies.

It is worth pointing out that dual-isotope imaging can also be applied to lung function, brain function, hyperparathyroidism and other clinical procedures and the strontium halide compositions of the present invention can be utilized in these applications (e.g., methods and systems) and studies in the future.

Scintillators for Single Photon Imaging

Scintillation crystals coupled to PMTs are commonly used as γ-ray detectors in single photon imaging. Table 2 provides a comparison of common inorganic scintillators considered in SPECT. Most commercial SPECT systems at present use NaI:Tl scintillators. NaI:Tl crystals are available in large sizes at reasonable cost and offer relatively high light output. The main limitation of NaI:Tl in SPECT imaging is its modest energy resolution (˜9% FWHM at 140 keV). CsI:Tl is a bright scintillator which also is available and cost-effective in large sizes. The spectral emission of CsI:Tl has a better match with silicon photodiodes than with PMTs and dedicated, single photon imaging systems for cardiac studies have been built using CsI:Tl scintillators with solid-state photodetectors (see, e.g., on the world wide web, at “digirad.com”). However, CsI:Tl scintillators also show relatively poor energy resolution at the photon energies used for SPECT (˜10% FWHM at 140 keV). For previously available scintillator compositions, such as both NaI:Tl and CsI:Tl, the energy resolution is limited by their nonproportional response. Scintillators such as YAP (YAlO₃:Ce) have been used in combined SPECT-PET small animal systems (e.g., on the world wide web, at “ise-srl.com/YAPPET/yap-doc.htm”). YAP:Ce shows a high degree of proportionality but its light output is low, which limits its energy resolution.

TABLE 2
Properties of Inorganic Scintillators for Nuclear Medicine

| | | Wavelength | Attenuation | Principal |
|---|---|---|---|---|
| | Light Output | of Maximum | Length (140 | Decay |
| Material | [Photons/MeV] | Emission [nm] | keV) [cm] | Time [ns] |
| NaI(Tl) | 38,000 | 415 | 0.38 | 230 |
| CsI(Tl) | 52,000 | 540 | 0.26 | 1000 |
| YAP | 20,000 | 370 | 0.65 | 26 |
| LaBr₃: Ce | ≧63,000* | 360 | 0.35 | 17 |
| SrI₂: Eu²⁺ | 80,0000- | 440 | 0.3 | ~1000 |
| | 120,000 | | | |

Saint Gobain quotes light yield of 63K photons/MeV for its LaBr₃: Ce crystals, present analysis has measured light output of 70K photons/MeV for its LaBr₃: Ce

Newer, rare earth trihalide scintillator such as LaBr₃:Ce show very high light output and fast response (see Table 2). LaBr₃:Ce scintillators have been reported to show high proportionality, though increased nonproportionality is reported at low electron energies. As a result, even though the energy resolution of LaBr₃:Ce is almost 2-fold better than that of NaI:Tl at 662 keV, the improvement at SPECT energies is rather modest. For example, at 140 keV, the energy resolutions of LaBr₃:Ce and NaI:Tl are ˜7% (FWHM) and 9% (FWHM), respectively, using production-grade PMTs with typical bialkali photocathodes. Also, large crystals of LaBr₃:Ce are still very expensive due to difficulties associated with growth of high quality, large crystals of LaBr₃ that are prone to cracking and cleavage. These problems arise mostly due to highly anisotropic, hexagonal structure of LaBr₃. The coefficient of thermal expansion for LaBr₃:Ce varies by a factor of three for its different crystallographic planes, which creates stresses in the crystal as it is cooled from its melting point. This has been reported as often leading to cracking and cleavage of the crystals during the cooling process. Also, LaBr₃ when heated to higher temperatures during crystal growth process is reported to form oxyhalides if any moisture or oxygen is present in the system. These oxyhalides can reduce yield of large, high quality LaBr₃ crystals. CeBr₃, a new rare earth halide scintillator, has scintillation properties similar to those for LaBr₃:Ce, as previously reported, and also faces many of the same challenges that are present for LaBr₃:Ce.

Also shown in Table 2 are some scintillation properties of SrI₂:Eu²⁺ as observed in preliminary studies (further optimization is also described herein). This scintillator shows very bright luminescence, higher than that for even LaBr₃:Ce. Furthermore, our recent studies indicate that SrI₂:Eu exhibits excellent proportionality over a wide energy range (as discussed in a later section). At low electron energies, the proportionality of SrI₂:Eu is higher than that of even LaBr₃:Ce, which indicates that SrI₂:Eu provides very high energy resolution for typical radioisotopes used in SPECT. Also, SrI₂ appears to be less vulnerable to oxyhalide formation and since it does not have a layered crystal structure, it is not prone to basal cleavage. These factors along with congruent melting of SrI₂ at low temperature indicate that growth of large crystals of SrI₂ from the melt using Bridgman and Czochralski methods is achievable.

Nonproportionality and Energy Resolution of Scintillators

As noted, scintillators need good proportionality to optimize their spectroscopic performance. Alkali-halide scintillators such as NaI:Tl and CsI:Tl, commonly used in SPECT and other gamma ray spectroscopy applications, are bright but have moderate energy resolution (˜6-7% FWHM for 662 keV photons). Significantly, the energy resolution of these alkali-halide scintillators is substantially worse than that expected from counting statistics (based on their light output). The measured energy resolution of most previously known scintillators lies considerably above a solid curve which represents the theoretical resolution (based on counting statistics), indicating that the energy resolution of most scintillators is worse than that predicted by counting statistics. It should also be noted that even small crystals of many previously known alkali-halide scintillators show poor energy resolution, which indicates that the degradation in energy resolution is not due to issues such as non-uniformity.

The present consensus is that the main cause for degradation in the energy resolution of common scintillators (such as CsI:Tl, Tl and LSO) is nonproportionality. The luminous efficiency (i.e. the number of scintillation photons per unit energy) of the scintillator depends on the energy of the particle that excites it. A gamma ray begins the excitation process by creating a knock-on electron either by photoelectric absorption or Compton scatter. As this primary electron traverses the scintillator, it loses energy to the scintillator (exciting it) and also produces other relatively high energy electrons (delta-rays), which also excite the scintillator. Thus, the scintillator is effectively excited by multiple electrons having a range of energies, even when the primary excitation source is a single gamma ray. If the luminous efficiency is independent of the electron energy, then the number of scintillation photons produced by two gamma rays with the same energy is the same (within counting statistics) because the sum of the electron energies is the same (and equal to the incident gamma energy). However, if the luminous efficiency depends on electron energy, then the number of scintillation photons will not necessarily be the same, and these variations degrade the energy resolution.

This dependence of luminous efficiency on electron energy has previously been measured using a Compton technique for commonly used/known alkali-halide scintillators. Ideally, the analysis should indicate no dependence on electron energy. None of the many previously known alkali-halides possess this ideal shape, and these materials which are significantly above the theoretical curve also possess significant nonlinearity. The luminous efficiency of other nonalkali halide scintillators such as LSO, BGO, GSO and BaF₂ also depend on electron energy. On the other hand, YAP has previously shown very proportional response and as a result, its measured energy resolution is close to the value predicted from photoelectron statistics. Unfortunately, the previously measured light output of YAP is low. Scintillators such as LaBr₃:Ce (and related rare earth trihalide compositions) have been shown to have good proportionality at high energies and as a result their measured energy resolution at 662 keV agrees well with its value predicted from photoelectron statistics. However, these scintillators have shown higher nonproportionality at lower electron energies. As a result, the measured resolution of LaBr₃:Ce using a PMT with standard bialkali photocathode at 140 keV is ≧7% (FWHM), which is poorer compared to the estimated value (based on photoelectron statistics) of ≦5% (FWHM).

Thus, in order to obtain high energy resolution with scintillators, it is important not only to have high light output and good uniformity, but also to have minimal dependence of the luminous efficiency on the electron energy (over a wide energy range). Based on discoveries as disclosed herein, SrI₂ doped with Eu²⁺ has been discovered such a material, having both high light output and excellent proportionality.

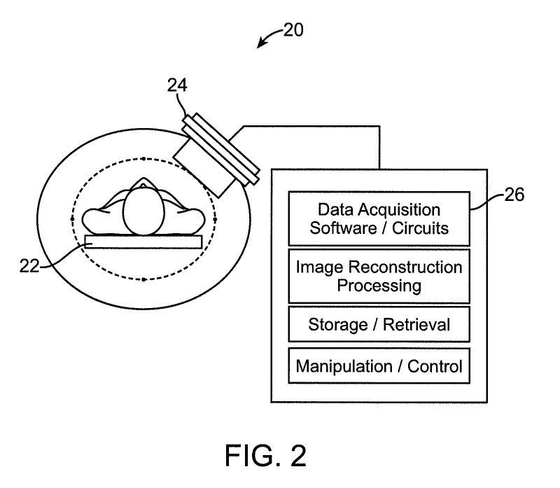

As discussed above, the scintillator compositions of the present invention are well suited for SPECT imaging, and the present invention will include SPECT imaging methods and systems including strontium halide scintillator compositions as disclosed herein. A basic configuration of a SPECT imaging system is described with reference to FIG. 2. The system 20 can include configurations/components commonly employed in known SPECT systems and further including strontium halide scintillator compositions. As shown, the SPECT system includes a patient or subject area 22 (e.g., positioned subject shown for illustrative purposes), a detector assembly 24 and a computer control unit 26. The computer control unit may include circuitry and software for data acquisition, image reconstruction and processing, data storage and retrieval, and manipulation and/or control of various components/aspects of the system. The detector assembly 22 can include a scintillator panel or area including a doped strontium halide scintillator material and a photodetector assembly optically coupled to the scintillator material. The system can include a single gamma-camera or detector in the detector assembly or a plurality of detectors, with various configurations and arrangements being possible. The detector assembly and subject area may be movable with respect to each other, and may include moving the detector assembly with respect to the subject area and/or moving the subject area with respect to the detector assembly. In use, radiation detection includes injecting or otherwise administering isotopes (including those commonly employed in SPECT imaging) having a relatively short half-life into the subject's body placeable within the subject area. The isotopes are taken up by the body and emit gamma-ray photons that are detected by the detector assembly. SPECT imaging is performed by using the detector assembly to acquire multiple images or projections (e.g., 2-D images), from multiple angles. The computer control unit is then used to apply image reconstruction and processing, e.g., using a tomographic reconstruction algorithm, to the multiple projections, yielding a 3-D dataset. This dataset may then be displayed as well as manipulated to show different views, including slices along any chosen axis of the body.

Scintillator compositions of the present invention can further be utilized in PET systems and imaging methods. In PET imaging, a PET imaging system detects pairs of gamma rays emitted indirectly by a positron-emitting radionuclide (tracer), which is introduced into the subject's body. Images of tracer concentration in 3-dimensional space within the body are then reconstructed by computer analysis. PET imaging systems and aspects of TOF PET imaging are further described in commonly owned U.S. Pat. No. 7,504,634, which is incorporated herein by reference in its entirety for all purposes.

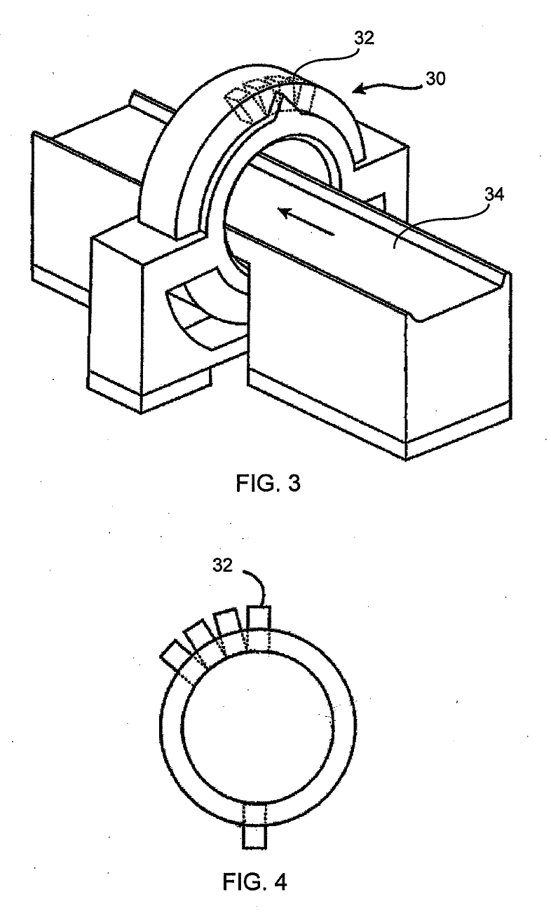

An exemplary basic configuration of a PET system according to the present invention is described with reference to FIG. 3. A PET camera system 30 includes an array of radiation detectors 32, which may be arranged (e.g., in polygonal or circular ring) around a patient area 34, as shown in FIG. 3. In some embodiments radiation detection begins by injecting or otherwise administering isotopes with short half-lives into a patient's body placeable within the patient area 34. As noted above, the isotopes are taken up by target areas within the body, the isotope emitting positrons that are detected when they generate paired coincident gamma-rays. The annihilation gamma-rays move in opposite directions, leave the body and strike the ring of radiation detectors 32.

As shown in FIG. 4, the array of detectors 32 includes an inner ring of scintillators, including compositions as presently described herein, and an attached ring of light detectors or photomultiplier tubes. The scintillators respond to the incidence of gamma rays by emitting a flash of light (scintillation) that is then converted into electronic signals by a corresponding adjacent photomultiplier tube or light detectors. A computer control unit or system (not shown) records the location of each flash and then plots the source of radiation within the patient's body. The data arising from this process is usefully translated into a PET scan image such as on a PET camera monitor by means known to those in the art.

In addition to gamma-ray imaging applications such as SPECT and PET, many, indeed most, ionizing radiation applications will benefit from the inventions disclosed herein. Specific mention is made to X-ray CT, X-ray fluoroscopy, X-ray cameras (such as for security uses), and the like.

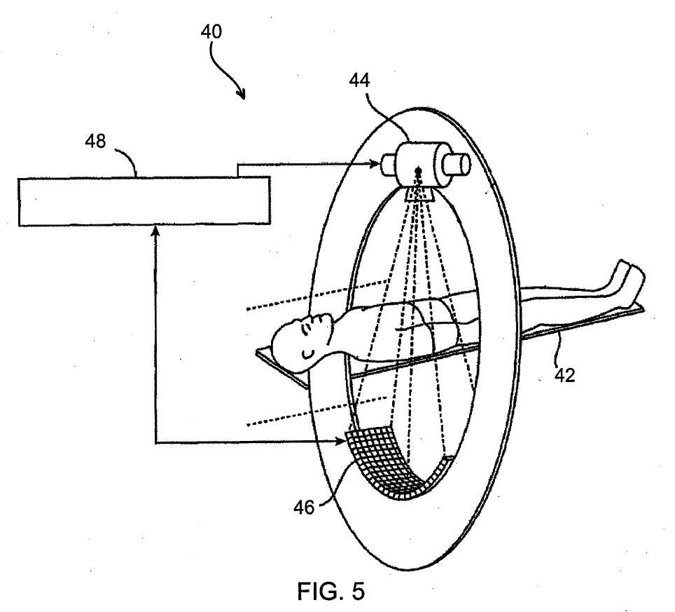

The present invention further includes CT imaging systems and methods, where scintillator compositions of the present invention will find use. A basic configuration of a CT scanner system is described with reference to FIG. 5, and can include configurations/components commonly employed in known CT systems. As shown, a CT system 40 includes a patient or subject area 42 (positioned subject shown for illustrative purposes), a penetrating X-ray source 44 (i.e., an X-ray tube), a detector assembly 46 and associated processing electronics, and a computer control unit 48, which may include circuitry and software for image reconstruction, display, manipulation, post-acquisition calculations, storage, data output, receipt, and retrieval. A detector assembly can include a scintillator panel or area including a doped strontium halide scintillator material and a photodetector assembly optically coupled to the scintillator material. The system further includes electronics (e.g., via computer control unit 48) coupled to the radiation detector assembly so as to output image data in response to radiation detection by the scintillator, including data storage, retrieval, processing, image reconstruction, and the like.

Systems and methods of the present invention as described above are illustrative, and alternate configurations and embodiments will be included. The present invention may include modifications as well as combinations of imaging systems as described, such as combined imaging systems—e.g., combined SPECT/x-ray CT systems, and the like.

Spectroscopic Applications at High Temperature

In another aspect, strontium halide scintillators of the present invention can be used for radiation detection at elevated or high temperatures. Strontium halide scintillators, such as strontium iodide containing compositions, demonstrate surprisingly high light output at high temperatures. Thus, the unexpected characteristic of strontium halide scintillators (e.g., SrI2) having excellent light output at high temperature, makes the scintillator compositions of the present invention suitable for high temperature radiation detection applications, such as well logging. High temperatures at which such radiation detection can be performed include, for example, average temperatures of the scintillator material or location at which radiation detection is performed in excess of 50 degrees C., and often in excess of 75 degrees C. Thus, high temperatures can range from about 50 degrees C. to about 200 degrees C. (e.g., including any number therebetween), or greater.

High temperature radiation detection according to the present invention can find use in a variety of contexts, including certain geological evaluation applications (e.g., subterranean radiation detection) where high temperature environments commonly are found. One of the uses in geological evaluation includes well logging or formation evaluation. Such well logging or formation evaluation studies can include measurement versus depth or time, or both, of one or more physical quantities in or around a well. Typically, a logging tool is lowered into a borehole and then retrieved from the well/hole while recording measurements. Wireline logs are taken “downhole”, transmitted through a wireline to the surface and recorded there. Measurement-while-drilling (MWD) and logging-while-drilling (LWD) measurements are also taken “downhole” or in a subterranean borehole. The measurements are either transmitted to the surface by mud pulses, or else recorded “downhole” and retrieved later when the instrument is brought to the surface. Mud logs that describe samples of drilled cuttings are taken and recorded at the surface.

Measurements typically taken during well logging or formation evaluation involve, for example, 1) natural gamma-ray spectroscopy to measure the spectrum or number and energy of gamma-rays emitted as natural radioactivity by a formation; 2) neutron activation which provides a log of elemental concentrations derived from the characteristic energy levels of gamma-rays emitted by a nucleus that has been activated by neutron bombardment; 3) epithermal neutron porosity measurement which is a measurement based on the slowing down of neutrons between a source and one or more detectors that measure neutrons at the epithermal level, where their energy is above that of the surrounding matter; 4) elastic neutron scattering which involves neutron interaction in which the kinetic energy lost by a neutron in a nuclear collision is transferred to the nucleus; and 5) gamma-ray scattering which is used for a measurement of the bulk density of a formation based on the reduction in gamma-ray flux between a source and a detector due to Compton scattering.

Scintillation and semiconductor detectors are typically used in these logging devices. It is known that the static temperature in a wellbore increases gradually with depth. In most of North America the increase or “gradient” will be between 0.5 and 2.5° F. for each 100 feet of increase in depth, with a value of 1.7° F./100 feet (3° C./100 meters) being typical. For these applications, one of the important characteristics of the detector is its ability to perform at high temperatures. Typical scintillators used in well logging devices include BGO and CsI(Tl) which perform poorly as temperature increases, losing half of their light output at around 75° C. and 130° C., respectively. SrI2 has a light output that increases with temperature.

Because the light output varies with temperature, for some spectroscopic applications, acquired or known data (see, e.g., Examples below) can be used to generate a calibration curve of light output versus temperature. Alternatively, a weak radioactive source such as Co-57 can be used to provide a known peak that can then be used to scale the spectra. The source can be on continuously, or it can be shuttered on and off between data acquisitions in situ. Alternatively, a light pulser can be used to provide a fixed reference signal.

The following examples are provided to illustrate but not limit the invention.

### EXAMPLES

### Example 1

### Preliminary Investigation of SRI₂:Eu and Related Scintillators

Overview:

In this section we present our recent investigation of strontium iodide doped with Eu²⁺ as a scintillation material. SrI₂ belongs to the alkaline-earth iodide family and it has orthorhombic structure. Some reports of scintillation from compositions belonging to alkaline-earth iodide family can be found in the literature, originating from the work of Hofstadter on calcium iodide in 1960's (Hofstadter 1964). Calcium iodide exhibits very high light yield (≧80,000 photons/MeV) and it can be activated with various dopants such as Tl⁺ and Eu²⁺. However, CaI₂ has hexagonal, layered crystal structure and is very prone to basal cleavage (Hofstadter 1964), which makes large volume growth of CaI₂ crystals very challenging. Hofstadter also reported scintillation from strontium iodide doped with Eu²⁺ with light yield approaching that for NaI:Tl (Hofstadter 1968), though very few subsequent publications can be found on this scintillator.

Since CaI₂:Eu has already shown excellent light yield (but faces difficulties in large volume growth due to its hexagonal structure that is prone to cleavage), upon optimization, other alkaline earth iodides such as SrI₂:Eu (which does not have layered crystal structure and therefore, do not cleave readily), may show scintillation performance similar to CaI₂:Eu without the associated difficulties in crystal growth. Crystals of SrI₂:Eu were grown by Bridgman method and scintillation properties of both compositions were evaluated.

Crystal Growth Aspects

SrI₂ is an orthorhombic composition belonging to alkaline-earth iodide family with density of 4.6 g/cm³, respectively. The melting point of SrI₂ is 540° C. In view of congruent melting of SrI₂ at low relatively low temperature, we produced crystals of this material using the Bridgman process. Evacuated quartz ampoules were used as crucibles in this study. Due to the orthorhombic crystal structure of these materials, crystal growth is expected to be relatively easy and our experimental work validated this expectation. SrI₂ and BaI₂ crystals (˜1 cm³ or larger, doped with 0.5% Eu²⁺ on molar basis) were produced in our preliminary study. Due to the hygroscopic nature of these materials, they need to be protected from prolonged exposure to moisture. Most r studies were performed in moisture free chambers or with protected crystals.

Scintillation Properties of SRI₂:Eu²⁺

Small crystals of SrI₂:Eu (<1 cm³ size, doped with 0.5% Eu²⁺ on molar basis) were cut and polished from Bridgman grown boules. These crystals were characterized using X and γ-rays to measure their emission and decay spectra as well as light output.

Emission Spectrum

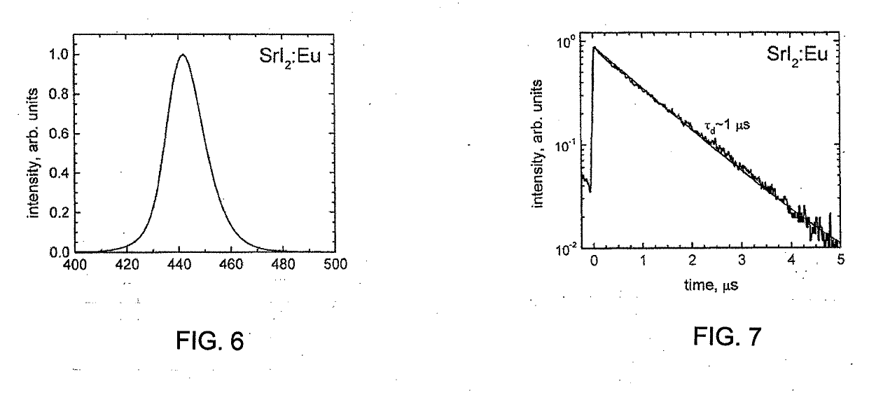

Optical emission spectrum of the SrI₂:Eu scintillator was measured. The crystal was excited with radiation from a Philips X-ray tube having a copper target, with power settings of 30 KVp and 15 mA. The scintillation light was passed through a McPherson 0.2-meter monochromator and detected with a Hamamatsu C31034 PMT with a quartz window. The system was calibrated with a standard light source to correct for sensitivity variations as a function of wavelength. FIG. 6 shows the emission spectrum for the SrI₂:Eu crystal. As seen in the figure, the emission from SrI₂:Eu occurs over a single, narrow band (420-480 nm), which is due to d→f transition of Eu²⁺. The wavelength of maximum emission (λₘₐₓ) is 440 nm for SrI₂:Eu. This wavelength matches well with the response function of PMTs as well as new Si-photodiodes.

Decay Time Spectrum

The fluorescent decay time spectrum of a SrI₂:Eu crystal under 511 keV gamma-ray excitation (²²Na source) was measured using the delayed coincidence method (Bollinger 1961) and the result is shown in FIG. 7. From an exponential fit to the data, the principal decay time constant was estimated to be ˜1 μs (which is attributed to Eu²⁺ luminescence). A single component fit is sufficient to cover all the recorded emission. Decay lifetime studies were also conducted using a flashlamp-pumped YAG:Nd laser using the 4^{th} harmonic at 266 nm (20 ns pulses). The temporal profile was fitted with a single component fit, and the decay time constant was estimated to be 1.2 μs. The overall decay time profile of SrI₂:Eu is adequate for SPECT and is similar to that for CsI:Tl which is already being used in single photon imaging studies.

Light Output and Energy Resolution

The light output of SrI₂:Eu crystal was measured. This study involved acquiring a ¹³⁷Cs gamma-ray spectrum (662 keV photons) with the SrI₂:Eu crystal using a Hamamatsu R980 bialkali PMT. The SrI₂:Eu scintillator, wrapped with several layers of Teflon tape, was coupled to the PMT using mineral oil. The PMT signal was shaped with Tennelec amplifier (TC 244) and shaping time of 4 μs was used. The pulse height spectra were then recorded with the Amptek MCA 8000-A multichannel analyzer. The measured photopeak was fit to a Gaussian to evaluate the peak position and full-width-at-half-maximum (FWHM) to estimate scintillation light yield and energy resolution, respectively. A pulse height spectrum was recorded first with SrI₂:Eu and then with a calibrated, packaged LaBr₃:Ce crystal with light yield of 60,000 photons/MeV (see also, below). From the measured 662 keV peak position for SrI₂:Eu and LaBr₃:Ce, and the known light output of the test LaBr₃:Ce crystal (60,000 photons/MeV), the light output of SrI₂:Eu was estimated to be ˜80,000 photons/MeV. This light yield is two times higher than NaI:Tl, the most common scintillator in SPECT systems and ˜30% higher than the LaBr₃:Ce crystal used in this study (Cherepy 2007), which is very encouraging. High light output is important in SPECT because in combination with proportionality of response, it governs energy resolution (and therefore, scatter rejection capabilities) of the scintillator. Furthermore, scintillators with high light output provide other practical benefits: larger PMTs can be used without any degradation in spatial resolution, which can significantly reduce the system cost.

The energy resolution of SrI₂:Eu²⁺ crystal at 662 keV was estimated to be ˜3.7% (FWHM) (see below). This energy resolution is two times higher than NaI:Tl and approaches that for LaBr₃:Ce. As crystals with higher optical quality are produced, we expect energy resolution to improve. Already at 122 keV (⁵⁷Co source), the energy resolution of SrI₂:Eu (<7% FWHM) is higher than that for LaBr₃:Ce with further improvement expected upon optimization of crystal quality and Eu²⁺ doping level, which bodes well for its use in SPECT studies.

Proportionality Studies

In addition to high light output, a scintillator needs to exhibit a highly proportional response in order to demonstrate high energy resolution. As discussed here, we have characterized proportionality of response of SrI₂:Eu using electron exposure.

The proportionality of SrI₂:Eu upon electron exposure has been measured using SLYNCI (Scintillation Light Yield Nonproportionality Characterization Instrument) (Cherepy 2007, Choong 2007b). In SLYNCI, a collimated 1 mCi Cs-137 source set 18 cm away illuminates the scintillator sample which is coupled to a Photonis PMT XP2060B (chosen due to its excellent linearity). The instrument employs five high-purity germanium (HPGe) detectors, each located at a different scattering angle 10 cm away from the scintillator sample under study, which measure the energy of scattered gamma rays detected in coincidence with Compton electron events in the scintillator as they are readout by the PMT (Hamamatsu R6231). The electron energy deposited in the scintillator for each event is calculated by subtracting the scattered gamma ray energy measured in the HPGe detector from the incident source energy (661.657 keV). Relative light yield as a function of electron energy for SrI₂:Eu, compared to that of NaI(Tl) and LaBr₃:Ce (see below). The proportionality of the light yield is excellent for SrI₂:Eu, and thus the contribution to energy resolution due to nonproportionality is expected to be small for SrI₂:Eu.

It is important to note that the proportionality of SrI₂:Eu at low electron energies is superior to that of even LaBr₃:Ce. This has important implications for its performance in SPECT imaging. As discussed earlier, when a gamma-ray interacts in a scintillator, it creates a knock-on electron either by photoelectric absorption or Compton scatter. As this primary electron traverses the scintillator, it loses energy to the scintillator (exciting it) and a cascade of electrons with varying energies are produced. Thus the scintillator is effectively excited by a large number of electrons with varying energies to create the scintillation pulse for a given event. The distribution of electron energies can change from event-to-event and as a result, if the electron response of scintillator is nonproportional, its energy resolution suffers. For a given input γ-ray energy, proportionality of the scintillator for all electron energies below that input energy governs its energy resolution. Since SPECT is conducted at relatively low γ-ray energies (80-250 keV, with emphasis on 140 keV), the proportionality of the scintillator at low electron energies has a greater effect on its measured energy resolution, which makes SrI₂:Eu very attractive.

The fact that SrI₂:Eu has high light output as well as excellent proportionality (even at low electron energies) bodes well for its future use in high resolution γ-ray spectroscopy studies. We believe that as the crystal growth processes are optimized and the optical quality of the crystals is improved, the energy resolution of SrI₂:Eu is significantly better. Ultimately, due to its high proportionality, as high quality crystals become available, the energy resolution of SrI₂:Eu most likely is dominated by photoelectron-statistics. As a result, our estimates indicate that energy resolution of <5% (FWHM) at 140 keV (^{99m}Tc) should be achievable with SrI₂:Eu, which would be extremely beneficial for scatter rejection in SPECT as well as in dual isotope studies.

TABLE 3
Properties of SrI₂: Eu in Measured Example

| | | | | 662 keV |
|---|---|---|---|---|
| | | Wavelength | Principal | Energy Reso- |
| | Light Output | of Maximum | Decay | lution |
| Material | [Photons/MeV] | Emission [nm] | Time [ns] | [%-FWHM] |
| SrI₂: Eu²⁺ | 80,000 | 440 | ~1000 | 3.7% |

### Example 2

The present example provides additional exemplary results from preliminary studies of europium-doped strontium iodide and corresponding scintillation characteristics. SrI₂(Eu) grown by the Bridgman method exhibited scintillation light yields (e.g., as high as 80,000 photons/Me). SrI₂(Eu) emits into a single narrow band, assigned to Eu²⁺, centered at 435 nm, with a decay time of 1.2 μs and it offers energy resolution better than 4% FWHM at 662 keV.

Detection sensitivity for weak gamma ray sources and rapid unambiguous isotope identification are principally dependent on energy resolution, and are also enhanced by high effective atomic number of the detector material. The inorganic scintillator currently providing the highest energy resolution is LaBr₃(Ce), ˜2.6% at 662 keV, but it is highly hygroscopic, possesses intrinsic radioactivity due to the presence of primordial ¹³⁸La and its crystal growth is still challenging. Strontium iodide doped with europium are candidate materials offering moderately high density, ρ=4.6, equivalent or higher light yields than LaBr₃(Ce) and no intrinsic radioactivity. The Eu²⁺ activator typically produces luminescence in the 410-450 nm region with a decay time of 300-1500 ns.

Reports of scintillation from the family of alkaline earth halides have been published, originating with the work of Hofstadter on calcium iodide in the 1960's. Calcium iodide exhibits light yields in the vicinity of 100,000 Photons (Ph)/MeV and has been activated with many dopants, including Tl⁺ and Eu²⁺; however, it is nearly impossible to grow substantial CaI₂ crystals due to its platelet growth habit. While Hofstadter patented the SrI₂(Eu) crystal in 1968 (Hofstadter 1968), no isotope-identifying devices based on this material appear to ever have been reported. A report on cathodoluminescence from Ca, Sr and Li halides described efficient Eu²⁺ activation and moderate hygroscopicity of these materials. Hence, in recent years this class of materials has been largely ignored for scintillation.

Strontium iodide was grown in quartz crucibles using the Bridgman method, as described above. Crystals described in this example were doped with 0.5 mole percent europium and were typically several cubic centimetres per boule, then cut into ˜1 cm³ pieces for evaluation.

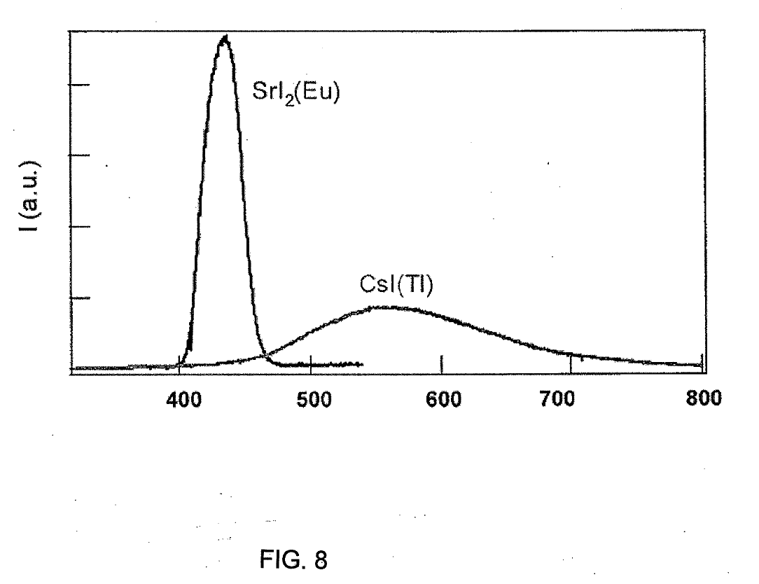

Radioluminescence spectra were acquired using a ⁹⁰Sr/⁹⁰Y source (average beta energy ˜1 MeV) to provide a spectrum expected to be essentially equivalent to that produced by gamma excitation. Radioluminescence spectra were collected with a Princeton Instruments/Acton Spec 10 spectrograph coupled to a thermoelectrically cooled CCD camera. The beta-excited luminescence of SrI₂(Eu), compared to that of a standard scintillator crystal, cesium iodide doped with thallium, is shown in FIG. 8. It possesses a single band centered at 435 nm, assigned to the Eu²⁺ d→f transition, and an integrated light yield approximately equivalent to that of CsI(Tl).

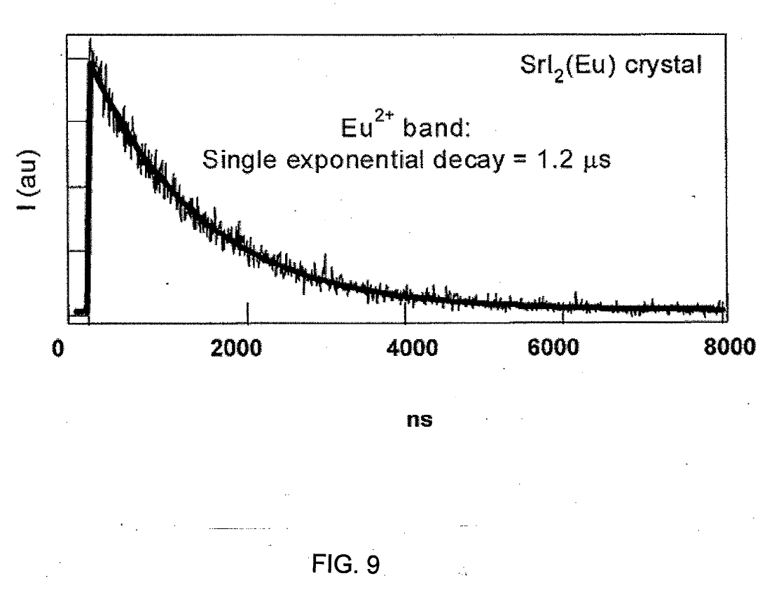

Decay lifetimes were acquired using a flashlamp-pumped Nd:YAG laser using the 4^{th} harmonic at 266 nm, and 20 ns FWHM pulses. Luminescence is collected with a monochromator coupled to an R928 Hamamatsu PMT and read out by an oscilloscope. In SrI₂(Eu), the Eu²⁺ band decays with a 1.2 microsecond time constant (FIG. 9).

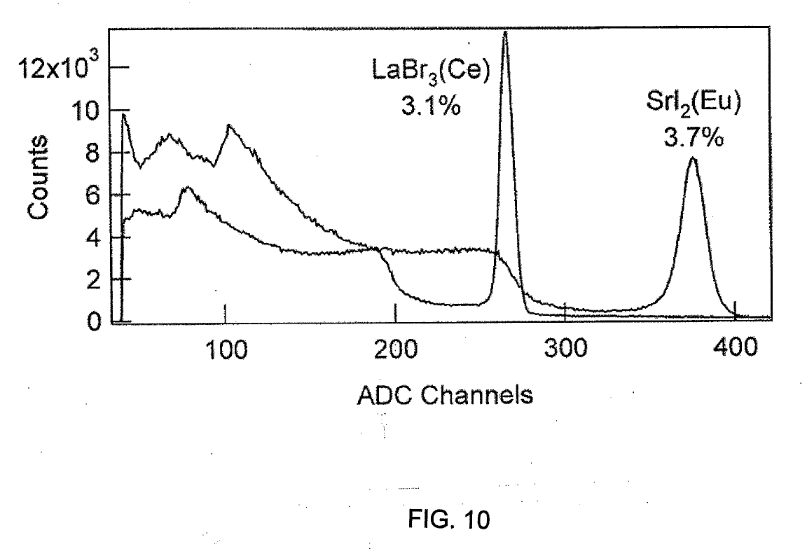

Gamma ray spectra were acquired using a Hamamatsu R980 bialkali PMT (spectral sensitivity in 380-420 nm range is nearly constant ˜30%). SrI₂(Eu) was optically coupled to the PMT by means of mineral oil and wrapped with several layers of Teflon tape. For all measurements, the scintillator was placed in the center of the entrance window of the PMT. The signals from the PMT anode were shaped with a Tennelec TC 244 spectroscopy amplifier (4 μs shaping time was used for SrI₂(Eu)) and then recorded with the Amptek MCA8000-A multi-channel analyzer. The total gamma absorption peaks (“photopeaks”) were fit to a Gaussian to evaluate the peak position and full width at half maximum (FWHM) to estimate the scintillation light yield and the energy resolution, respectively. Gamma light yields are determined by direct comparison of the photopeak position for SrI₂(Eu) and LaBr₃(Ce) (assumed to have a light output of 60,000 ph/MeV), since the spectral sensitivity of the bialkali photocathode is constant in the range of their luminescence. FIG. 10 shows the pulse-height spectra acquired using the 662 keV gamma from ¹³⁷Cs for SrI₂(Eu) and LaBr₃(Ce) under the same conditions. Energy resolution at 662 keV of <4% and light yield significantly superior to that of LaBr₃(Ce) are reproducibly measured for SrI₂(Eu).

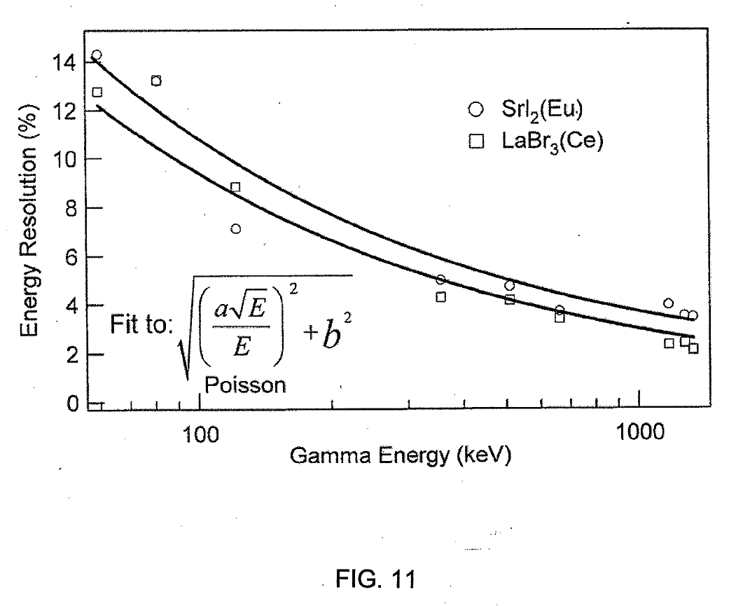

FIG. 11 shows the energy resolution as a function of gamma ray energy for SrI₂(Eu) and LaBr₃(Ce) using Ba-133, Am-241, Co-57, Na-22, Co-60 and Cs-137 sources. A fit to the experimental points using Poisson statistics form with an offset, shown in FIG. 11, indicates that the deviation from ideal behaviour is greater for SrI₂(Eu), since the crystal uniformity, optical quality, geometry and reflector configuration have not yet been optimized.

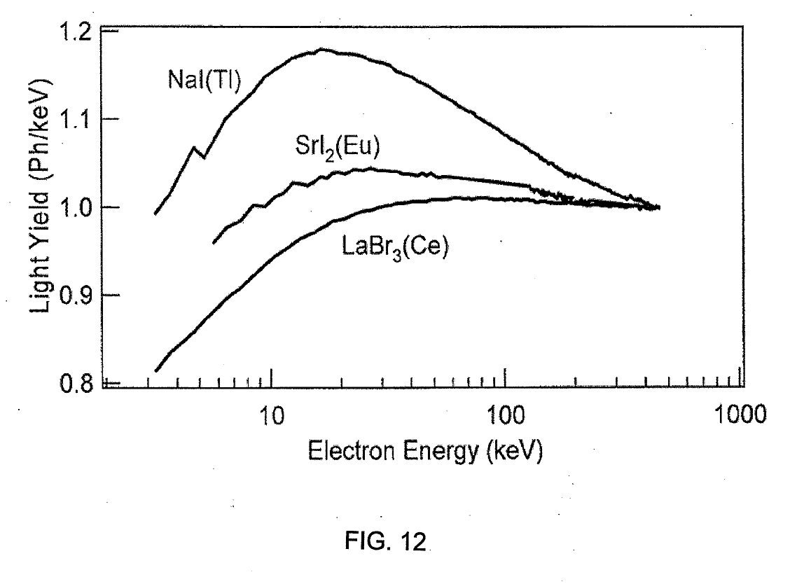

The design of the SLYNCI (“scintillation light yield nonproportionality characterization instrument”) is described, for example, in one or more of the cited references. It is a unique facility for measuring the so-called nonproportionality of scintillator materials. A collimated 1 mCi Cs-137 source set 18 cm away illuminates the scintillator sample which is coupled to a Photonis PMT XP2060B (chosen due to its excellent linearity). The instrument employs five high-purity germanium (HPGe) detectors, each located at a different scattering angle 10 cm away from the scintillator sample under study, which measure the energy of scattered gamma rays detected in coincidence with Compton electron events in the scintillator as they are readout by the PMT (Hamamatsu R6231). The electron energy deposited in the scintillator for each event is calculated by subtracting the scattered gamma ray energy measured in the HPGe detector from the incident source energy (661.657 keV). FIG. 12 shows the relative light yield as a function of electron energy for SrI₂(Eu), compared to that of NaI(Tl) and LaBr₃(Ce). The proportionality of the light yield is excellent for SrI₂(Eu). Future experiments will be conducted to verify this expectation.

These preliminary results indicated that Strontium iodide is a readily growable crystal that activates efficiently with Eu²⁺, which yielded in the present example a light yield of at least up to 80,000 photons/MeV and demonstrating <4% energy resolution at 662 keV. Its energy resolution and light yield proportionality surpass that of NaI(Tl) and approach those of LaBr₃(Ce). Improved results are expected upon further optimization the crystal uniformity, light collection and readout.

### Example 3

Energy resolution studies of scintillator compositions are further described below.

The overall energy resolution of a scintillator-PMT spectrometer (ΔE/E or R) can be expressed as follows (van Eijk 2001):

(ΔE/E)²=R²=R_{lid}²+R_{sci}²+Rₙₒᵢₛₑ² (Equation No. 1)

R_{lid} represents contribution for a light detection mechanism involving an ideal light source and an ideal photodetector. R_{sci} represents broadening effects due to non-ideal nature of scintillators. This parameter includes contribution of effects such as inhomogeneities, imperfect scintillator-photodetector coupling, and nonproportionality. Finally, the noise effects in the scintillation detection system are included in the final term, Rₙₒᵢₛₑ. For a given energy spectrum, the number of photoelectrons (Nₚₕₑ) corresponding to the measured peak position and the variance of electron multiplication gain of PMT (ε, which is ˜0.15) can be used to estimate R_{lid} using the expression:

R_{lid}²≈(2.36)²(1/Nₚₕₑ)(1+ε) (Equation No. 2)

For good coupling between the photodetector and the scintillator, the number of photoelectrons (Nₚₕₑ) corresponding to the measured peak position and R_{lid} can be expressed as follows:

Nₚₕₑ≈L·E·η (Equation No. 3)

R_{lid}²≈(2.36)²(1/(L·E·η))(1+ε) (Equation No. 4)

Where L is the light output of the scintillator in photons/MeV, E is the gamma-ray energy in MeV and η is the quantum efficiency of the photodetector over the optical emission spectrum of the scintillator. For PMTs, Rₙₒᵢₛₑ is negligible, though for silicon photodetectors the detector and electronic noise components are included in this parameter. Based on this, the energy resolution of a scintillation with PMT readout can be expressed as:

R²=R_{lid}²+R_{sci}²≈(2.36)²(1/(L·E·η))(1+ε)+R_{sci}² (Equation No. 5)

C. W. E. van Eijk et al. have performed such an analysis of the energy resolution of a NaI(Tl) scintillator coupled to a PMT for 662 keV photopeak (see Table 4). The overall energy Resolution® at 662 keV energy was measured to be 6.7% (FWHM) for NaI(Tl) coupled to PMT. R_{lid} was estimated to be 3.2% (FWHM) using Equation No. 4. R_{sci} was then calculated to be 5.9% (FWHM) from Equation No. 5. This analysis illustrates that the non-ideal nature of scintillator (represented by the term R_{sci}) is the dominant resolution broadening component for NaI(Tl), which can be explained on the basis of highly nonproportional response of NaI(Tl) (see above). In past, we have performed similar analysis of the energy resolution of LaBr₃:Ce at 662 keV (see Table 4). The measured energy resolution of LaBr₃:Ce at 662 keV was 3% (FWHM), while R_{lid} for LaBr₃-PMT detector was calculated to be 2.3% (FWHM). R_{sci} was then estimated to be 1.9% (FWHM). This study indicates that photoelectron statistics (or R_{lid}) is the main broadening component for LaBr₃:Ce, while the contribution of the term R_{sci} (representing the non-ideal nature of scintillator) is much lower. This reduction in R_{sci} in case of LaBr₃:Ce can be explained by its significantly higher proportionality than NaI(Tl) (see above).

TABLE 4
Analysis of the 662 keV Energy Resolution of
Scintillator-PMT Spectrometers

| | Nₚₕₑ | | | | |
|---|---|---|---|---|---|
| Detector | (at 0.662 MeV) | R (%) | R_{lid} (%) | R_{sci} (%) | Rₙₒᵢₛₑ(%) |
| NaI(Tl)-PMT | 6,000 | 6.7 | 3.2 | 5.9 | 0 |
| LaBr₃:Ce-PMT | 10,350 | 3 | 2.3 | 1.9 | 0 |

Since SrI₂ is a very bright and proportional scintillator, it is expected that its energy resolution is dominated by photoelectron statistics (R_{lid}) and not by the non-ideal nature of the scintillator (R_{sci}) It is worth noting that at 140 keV, the energy resolution broadening estimated from term R_{lid} (expected to be the dominant term) is ˜4.7% (FWHM) for optical readout using PMTs with standard bialkali photocathodes (QE˜0.25), indicating that overall resolution below 5% (FWHM) should be achievable even if some non-idealities are present. If silicon p-i-n photodiodes (QE˜0.7) or PMTs with new ultra-bialkali photocathodes (QE˜0.43) are used, R_{lid} can be 2.8% (FWHM) and 3.6% (FWHM), respectively, which is very encouraging. For silicon p-i-n photodiode readout some detector cooling may be needed to reduce the electronic noise due to dark current in the detectors.

### Example 4

In this example, additional analysis exemplary SrI₂ scintillator compositions and properties is described. The effect of dopant concentration was further examined, and even higher light output totals and improved energy resolution were observed compared to some preliminary investigations. These results indicated that SrI₂ scintillator compositions of the invention are among the most promising scintillators in existence for nuclear spectroscopy applications. Additionally, SrI₂ doped with Ce³⁺/Na⁺ is investigated and found to have a much faster response, though it is yet unable to match the high light output totals of SrI₂:Eu2⁺. In this example, crystal growth techniques as well as the effect of dopant concentration on the scintillation properties of SrI₂, over the range 0.5% to 8% Eu²⁺ and 0.5% to 2% Ce³⁺/Na⁺, are described.

SrI₂:Eu²⁺ and SrI₂:Ce³⁺/Na⁺ crystals were grown using the vertical Bridgman method. The nominal Eu2+ concentration was: 0.5, 2, 5, and 8% (by mole). The crystals were grown in silica ampoules using Anhydrous SrI₂ beads (Aldrich, 99.99%), EuI₂ powder (Aldrich, 99.9%), CeI₃ beads (Aldrich, 99.99%), and NaI beads (Aldrich, 99.999%) as the starting materials. The ampoules were loaded in an inert gas environment. Using a Varian Vac Sorb pump, the ampoules were evacuated to ˜10⁻³ Torr and heated to 150° C. to ensure that all moisture was eliminated. Next, by wrapping them in a wet towel, the crystals were kept cool as to prevent thermal decomposition while a torch was used to seal the ampoules. Vertical Bridgman furnaces were used to grow the crystals, with the ampoules lowered through the hot zone at 10 mm/day. The melting point of SrI₂ is ˜538° C. and the hot zone temperature was set to 588° C.

Single crystals included those of size 10 mm in diameter by 40 mm long. The crystal density, based on lattice parameters, is 4.59 g/cm3 and has a Z_{eff} of 50. SrI₂:Eu²⁺ crystals grown have proven to be optically clear and show minimal Eu2+ segregation. SrI₂:Ce³⁺/Na⁺ had shown some Ce³⁺ segregation, particularly the 2% crystal, which has a green coloration and was generally of a poorer optical quality. The SrI₂:0.5% crystal was of good optical quality and Ce³⁺ segregation seems relatively minimized. From these single crystals, a variety of samples ranging from 0.25 to 1.5 cm3 were prepared by cutting them from the solid ignots and polishing them using nonaqueous slurries. The crystals are highly hygroscopic and care was taken in handling them.

Scintillation properties of the SrI₂:Eu²⁺ and SrI₂:Ce³⁺/Na⁺ crystals were characterized, including measurements of the light output, emission spectrum, and the fluorescent decay time of the crystals as well as analysis of the pulse height spectrum under gamma excitation. Variation of these scintillation properties with Eu²⁺ concentration was also analyzed. A summary of certain scintillation properties of these crystals is included in Table 1 above; corresponding properties of LaBr₃:Ce³⁺ are included for reference.

Emission

Radioluminescence spectra were recorded with a Philips X-ray tube having a Cu anode operated at 40 kV and 20 mA. The scintillation light was dispersed through a McPherson 234/302 monochromator and subsequently detected with a Hamamatsu R2059 photomultiplier tube (PMT). FIG. 13 shows the radioluminescence spectra of SrI₂:Eu²⁺, SrI₂:0.5% Ce³⁺/Na⁺ and SrI₂:2% Ce³⁺/Na⁺, normalized to their respective maxima.

The spectrum of SrI₂:5% Eu²⁺ includes of a broad band peaking at approximately 430 nm emanating from the Eu²⁺ d→f transition. This emission spectrum does not differ in a statistically significant way from SrI2 crystals with different Eu²⁺ dopant concentrations. The spectra of the SrI₂:Ce³⁺/Na⁺ crystals show two components, typical of the 5d→4f, at 404 nm and 434 nm. The ratio of the intensities of the 404 nm peak to the 434 nm peak is decreased when the dopant concentration is increased from 0.5% to 2%.

Time Profiles

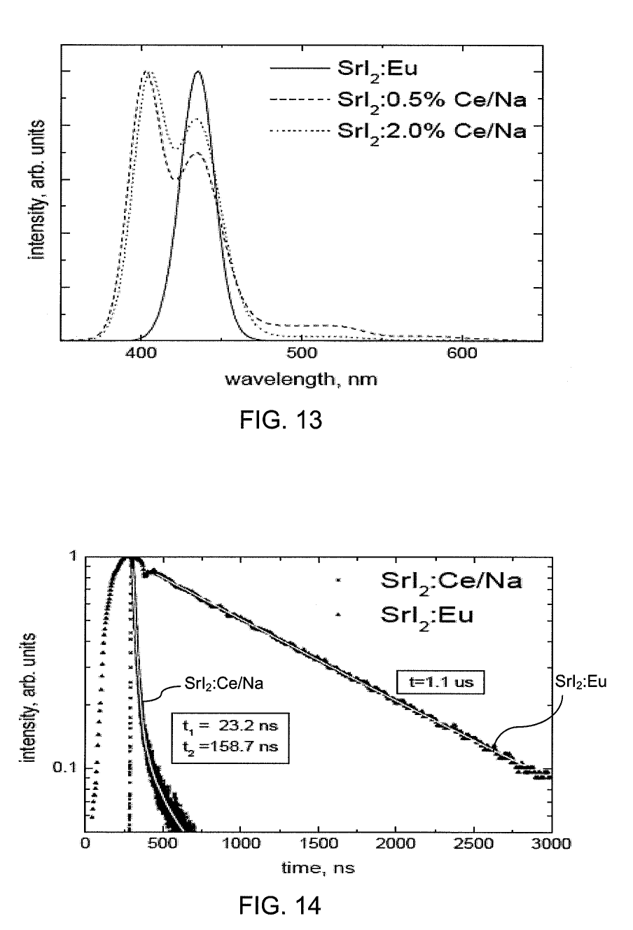

Scintillation decay time spectra for SrI₂:Eu²⁺ were recorded using a ¹³⁷Cs gamma ray source and a Tektronix TDS 220 oscilloscope connected to the output of a Hamamatsu R2059 PMT. The decay profile of SrI₂:0.5% Eu²⁺, shown in FIG. 14, exhibits a single decay component of 1.2 μs. The decay time of SrI₂:Eu²⁺ remains unchanged with different Eu²⁺ concentration levels.

The time profile of SrI₂:Ce³⁺/Na⁺ was recorded via the Bollinger method using two Hamamatsu R2059 PMTs. The SrI₂:2% Ce³⁺/Na⁺ time profile possesses a 23 ns fast component and a 159 ns slow component. The rise time of SrI₂:0.5% Ce is 1.15 ns. The principal decay component of SrI₂:0.5% Ce³⁺/Na⁺ contributes 47% of the total light emitted. Decay traces for SrI₂:0.5% Eu2+ and SrI₂:2% Ce³⁺/Na⁺ are shown in FIG. 14. The decay time recorded for the SrI₂:2% Ce³⁺/Na⁺ crystal showed two components, one of 33 ns and the other of 570 ns, with 46% of the light coming from the fast decay component and 54% from the slower component. The fast decay time of SrI₂:Ce³⁺/Na⁺ suggests suitability for use in timing applications. However, improvement of the light yield is desired. Improved crystal quality at higher Ce³⁺/Na⁺ levels should improve the light output totals of SrI₂:Ce³⁺/Na⁺; in which case the Ce³⁺/Na⁺ doped crystal could prove useful for fast timing applications, such as time-of-flight PET imaging.

Afterglow, produced under x-ray excitation, was also recorded at longer time scales to get a measure of the afterglow of SrI₂:Eu²⁺. For this a special apparatus was used including a 60 kW Electromed CPX160 x-ray generator with a Varian rotating anode tube (model A292), capable of providing square pulses ranging in length from 1 ms to 8 s, over a similarly wide range of tube voltages and currents. The scintillation signal is detected by a fast-response silicon PIN photodiode made by Hamamatsu, model S3204-8.

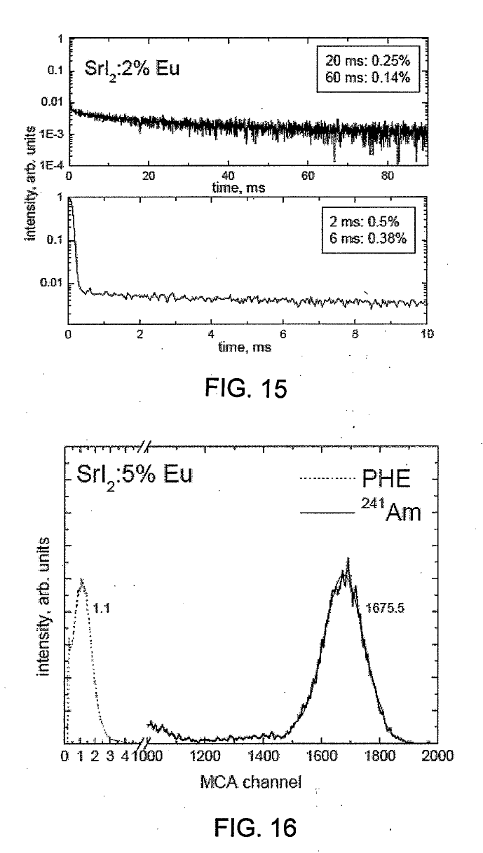

SrI₂:2% Eu²⁺ was found to decay to 0.5% of its maximum intensity after 2 ms. The time profile recorded in these measurements is shown in FIG. 15. After 6 ms the signal intensity has decayed to 0.38% of the maximum intensity. It is at 0.25% after 20 ms and 0.14% 60 ms after excitation. These levels of afterglow are comparable to those of co doped CsI:Tl⁺/Eu²⁺, a scintillator that has shown promise in high-speed imaging, an application where minimal afterglow is critical.

Light Output and Energy Resolution

The light output for each dopant concentration has been measure using a single photoelectron method. FIG. 16 shows the photopeak recorded under ²⁴¹Am irradiation as well as the single photoelectron peak. SrI₂:5% exhibits very high light output of over 120,000 photons/MeV. The light output levels of SrI₂:0.5% Eu²⁺, SrI₂:2% Eu²⁺, and SrI₂:8% Eu²⁺ are 68,000, 84,000, and 80,000 photons/MeV, respectively. These are some of the highest light output totals ever observed from inorganic scintillators. These results suggest that 5% Eu²⁺ may be the optimal dopant quantity. However, higher light output totals for SrI₂:0.5% Eu²⁺ have been observed, so it is possible there is room for improvement in these light yield totals for different samples.

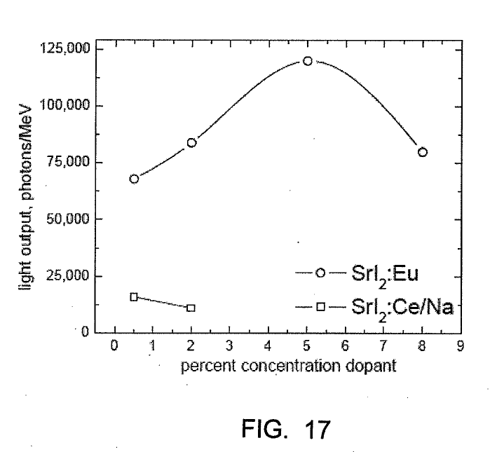

SrI₂:0.5% Ce³⁺/Na⁺ exhibited a light output of 16,000 photons/MeV, while SrI₂:2% Ce³⁺/Na⁺ yielded 11,000 photons/MeV. The SrI₂:0.5% Ce³⁺/Na⁺ was of much better optical quality than the SrI₂:2% Ce³⁺/Na⁺ crystal. It can be reasonably expected that the light output total will improve with crystal quality. The light output totals as a function of dopant concentration are shown in FIG. 17.

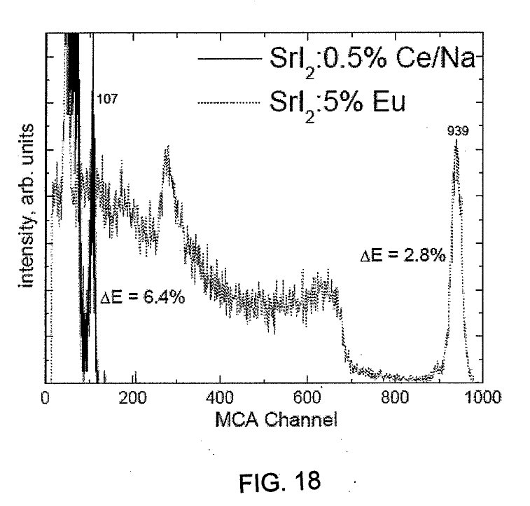

The pulse-height spectrum of SrI₂:5% Eu²⁺ under ¹³⁷Cs excitation was recorded with the crystal coupled to a Hamamatsu R6233-100 PMT with a super bialkali photocathode. The signal was shaped using a Canberra 2022 spectroscopy amplifier [4 μs shaping time] and transferred to an Amptek 8000A multichannel analyzer. The recorded spectrum is shown in FIG. 18. The quantum efficiency of the PMT is ˜21% for the emission profile of SrI₂:Eu²⁺. The crystals were sealed in a quartz cylinder with the sides and top wrapped in Teflon tape. The remaining exposed face was then coupled to the PMT with optical grease. The crystals of other dopant concentrations were recorded on a Hamamatsu R6233 PMT.

Upon fitting the resultant gamma absorption peak with a Gaussian function, SrI₂:5% Eu²⁺ showed an energy resolution of 2.8% at 662 keV. The energy resolutions at 662 keV of SrI₂:0.5% Eu²⁺, SrI2:2% Eu²⁺, and SrI₂:2% Eu²⁺ are 5.3%, 3.9%, and 4.9%, respectively. The energy resolution of SrI₂:5% Eu²⁺ is competitive with any commercially available scintillator.

SrI₂:0.5% Ce³⁺/Na⁺ showed an energy resolution of 6.4% at 662 keV while SrI₂:2% Ce³⁺/Na⁺ showed 12.3% energy resolution. The energy resolution recorded for SrI₂:0.5% Ce³⁺/Na⁺ accurately represents the capability of the crystal. However, the poor optical quality of the SrI₂:2% Ce³⁺/Na⁺ crystal suggests that better energy resolution can be expected.

Proportionality of Response

The light yield over a range of gamma ray excitation energies was characterized by inspection of the pulse height spectra recorded under excitation by various radioisotopes. The isotopes used were ⁵⁷Co (14 keV, 122 keV), ²⁴¹Am (60 keV), ¹³⁷Cs (662 keV), and ²²Na (511 keV, 1274 keV).

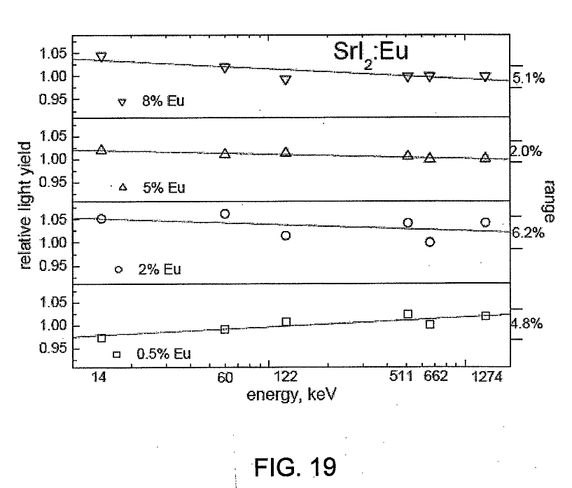

The relative light yield (per keV) of SrI₂:5% Eu²⁺ and SrI₂:Ce³⁺/Na⁺ as a function of gamma ray energy is shown in FIG. 19. SrI₂:5% Eu2+ demonstrates a remarkably linear response; with a deviation of less than 2% over the energy range from 14 keV to 1274 keV. SrI₂:2% Ce³⁺/Na⁺ also proved to be very linear in its response with a deviation of less than 6% over the range 60 keV to 1274 keV. For reference, the non-proportionality of LaBr₃:Ce³⁺ over the smaller range 60 keV to 1274 keV is 4%. This high level of linearity in SrI₂:Eu²⁺ indicates that the negative contribution to energy resolution from non-proportionality, relative to other scintillation materials, is minimized.

In summary, Strontium iodide is a crystal that is efficiently activated with Eu²⁺. SrI₂:Eu²⁺ shows excellent energy resolution of 2.8% at 662 keV, owing to its high light output, observed in the present example at up to 120,000 photons/MeV, and very linear response over a wide range of energies. Such properties place these crystals among the best inorganic scintillators for gamma ray spectroscopy, rivaling LaBr₃:Ce³⁺. SrI₂:Ce³⁺/Na⁺ does not have the high light output of the Eu²⁺ doped crystals, but its fast principal decay component of 25.2 ns suggests suitability for fast timing applications, particularly when considering the room for improvement as crystal quality is improved and Ce³⁺/Na⁺ concentration is increased.

The results of this study suggests that 5% Eu²⁺ is near the optimal dopant concentration. Further studies of concentrations near that range are necessary as well as fabrication of additional samples with the same dopant concentration levels investigated here to ensure that the results are not indicative of sub-optimal crystal quality. Improvements in crystal growth, handling, and packaging should lead to further improvement of these scintillators, which already demonstrated excellent performance.

### Example 5

In this example, additional analysis of exemplary SrI₂ scintillator compositions doped with thallium (Tl) and corresponding properties are described. Crystals were grown using the Bridgman method as described above. Crystals were grown included SrI₃:TlI/YI₃ (e.g., YI₃ as a compensator), with dopant concentrations of 0.5% and 2%. Radioluminescence spectra, scintillation decay time spectra, and proportionality of response were recorded as described above. The results indicate that SrI₂ doped with Tl provide useful scintillator compositions exhibiting peak radioluminescence in a useful range, good proportionality, and fast decay time.

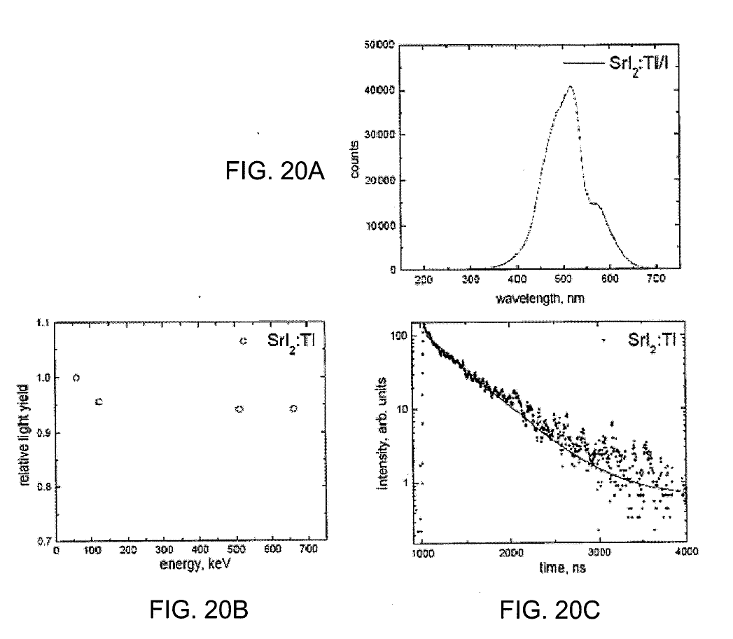

FIG. 20A shows the radioluminescence spectrum of SrI₂ (Tl/I). The spectrum includes a b and peaking at approximately 525 nm. Relative light yield (per keV) of SrI₂ (Ti/I) as a function of gamma ray energy is shown in FIG. 20B. As shown, SrI₂ (Tl/I) demonstrates a good linear response. A decay trace for SrI₂ (Tl/I) is shown in FIG. 20C. Principle decay time was measured at about 500 ns.

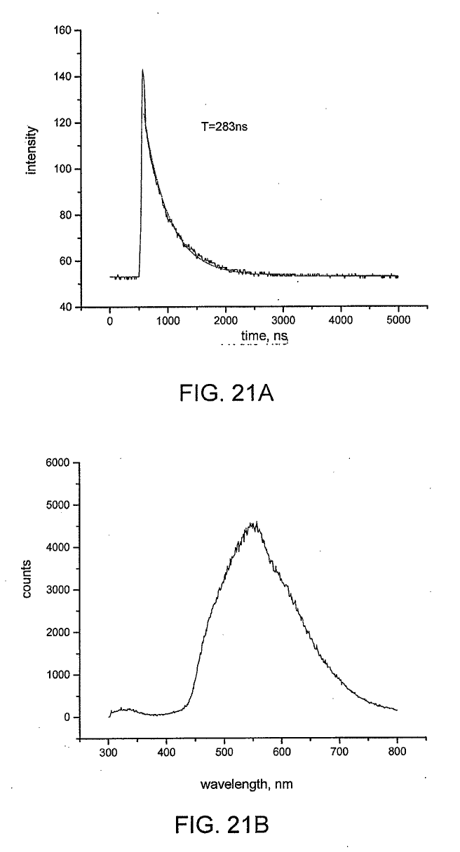

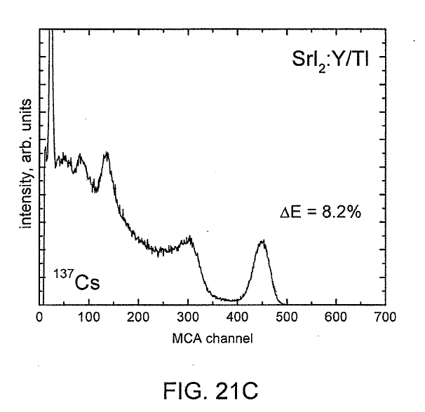

FIG. 21A shows a decay trace for SrI₃:TlI/YI₃ with a principal decay time measured at about 283 ns. FIG. 21B shows a radioluminescence spectrum of SrI₃:TlI/YI₃, with peak emission centered at about 550 nm. FIG. 21C shows an energy spectrum for SrI₃:TlI/YI₃, with energy resolution at about 8.2% at 662 keV.

### Example 6

Scintillation characteristics of SrI₂ scintillator compositions were examined at a range of temperatures so as to examine the effect of temperature on composition scintillation characteristics. Results indicated that SrI₂ scintillator compositions are suitable for use in radiation detection applications performed at elevated temperatures.

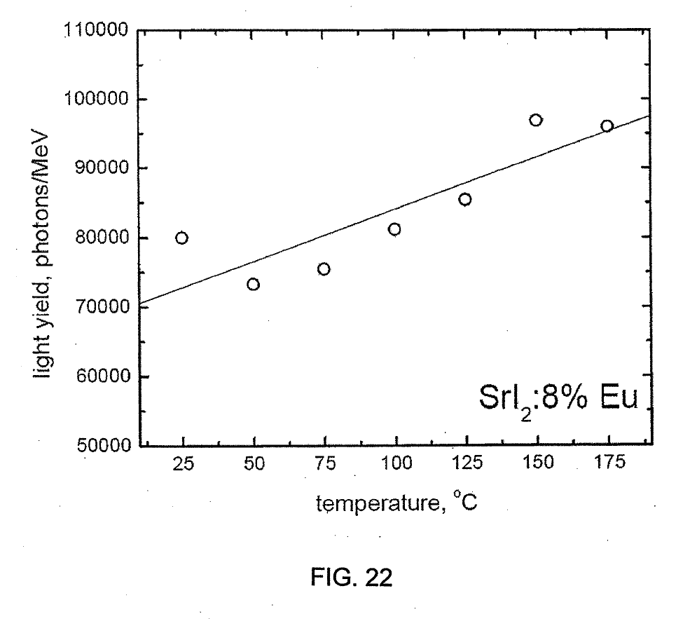

Crystals were grown using the Bridgman method as described above. SrI₂ doped with 8% Eu were specifically examined. Suitability of strontium halide scintillation compositions of the present invention for radiation detection at elevated or high temperatures is illustrated with reference to FIG. 22. As illustrated, FIG. 22 shows the light output of SrI2(8% Eu) as a function of temperature in the range of 25 to 175 C. As can be seen, the already high light output increases with temperature in this range.

Although the invention has been described with reference to the above examples, it will be understood that modifications and variations are encompassed within the spirit and scope of the invention. Accordingly, the invention is limited only by the following claims along with their full scope of equivalents.

What is claimed is:

1. An imaging system, comprising:
a subject area;
a radiation detection assembly comprising a doped strontium iodide scintillator material and a photodetector assembly optically coupled to the scintillator material; and
electronics coupled to the radiation detection assembly so as to output image data in response to radiation detected by the scintillator.

2. The system of claim 1, wherein the imaging system is a computed tomography system, X-ray computed tomography system, or a single photon emission computed tomography (SPECT) system.

3. The system of claim 1, wherein the radiation detected by the scintillator comprises gamma rays emitted from a radiopharmaceutical label administered to a subject positioned in the subject area.

4. The system of claim 1, wherein the dopant comprises europium.

5. The system of claim 1, wherein the dopant comprises cerium or thallium.

6. The system of claim 1, wherein the dopant is present at less than about 20% by molar weight.

7. The system of claim 1, wherein the dopant is present at between about 0.01% to about 10% by molar weight.

8. The system of claim 1, wherein the scintillator material comprises a crystalline, ceramic, or polycrystalline ceramic form.

9. The system of claim 1, wherein the photodetector assembly comprises a photomultiplier tube, a photodiode, a PIN detector, a charge-coupled device, or an avalanche detector.

10. The system of claim 1, further comprising a computer control system coupled to the detection assembly so as to receive, output, or process the image data, or comprising instructions for operation of the system.

11. A method of performing imaging of a subject using the system of claim 1.

12. A method of performing imaging of a subject, comprising:
positioning a subject in a patient area, wherein the patient has been administered with a radiopharmaceutical label;
positioning a radiation detection assembly adjacent to the subject, the detection assembly comprising a europium doped strontium iodide scintillator material and a photodetector assembly optically coupled to the scintillator material;
detecting gamma ray emissions from the patient with the radiation detection assembly so as to generate subject image data.

13. A scintillator composition comprising a Tl or Ce doped strontium halide scintillator.

14. The scintillator composition of claim 13, wherein the scintillator is a thallium-doped strontium iodide scintillator.

15. A method of performing radiation detection at a high temperature location, comprising:
positioning a radiation detection assembly in a high temperature area, the assembly comprising a doped strontium iodide scintillator material and a photodetector assembly coupled to the scintillator material; and
detecting radiation emissions from a radiation source in the high temperature area.

16. The method of claim 15, wherein the high temperature area comprises an average temperature exceeding 50 degrees C.

17. The method of claim 15, wherein the high temperature area comprises an average temperature of greater than about 75 degrees C. to greater than about 200 degrees C.

18. The method of claim 15, wherein the high temperature area comprises a wellbore or a subterranean location.

19. The method of claim 15, wherein the radiation detection comprises a well logging or geological formation evaluation.

20. The method of claim 15, wherein the radiation emissions comprise gamma-ray or neutron emissions.

21. The method of claim 15, further comprising providing calibration data comprising one or more scintillation characteristics of the scintillator composition as a function of temperature; and scaling a detected radiation emission spectra from the high temperature environment relative to the calibration data.

22. The method of claim 21, wherein the one or more scintillation characteristic comprises light output.

23. The method of claim 21, wherein the calibration data comprises measured light output versus temperature.

24. The method of claim 21, wherein the calibration data is generated by recording radiation from a radiation source placed proximate to the detector, and the recording comprises continuously recording said source radiations or shuttering on and off radiation pulses during data acquisition.

25. The method of claim 21, further comprising providing a light pulser so as to provide a fixed reference signal.
